(19) United States
(12) Patent Application Publication
(10) Pub. No.: US 2016/0045625 A1
(43) Pub. Date: Feb. 18, 2016

(54) [11C] AND [18F] LABELED 1,3-DIPHENYL-5-(PYRIMIDIN-2-YL)-PYRIDIN-2(1H)-ONE DERIVATIVES AND THEIR USE FOR PET IMAGING OF THE AMPA RECEPTOR

(71) Applicants: EISAI R&D MANAGEMENT CO., LTD., Bunkyo-ku, Tokyo (JP); NATIONAL INSTITUTE OF RADIOLOGICAL SCIENCES, Chiba-shi, Chiba (JP)

(72) Inventors: Norihito Oi, Tsukuba (JP); Noboru Yamamoto, Tsukuba (JP); Michiyuki Suzuki, Tokyo (JP); Yosuke Nakatani, Tsukuba (JP); Tetsuya Suhara, Chiba (JP); Meiei Cho, Chiba (JP); Toshimitsu Fukumura, Chiba (JP); Makoto Higuchi, Chiba (JP); Takafumi Minamimoto, Chiba (JP); Jun Maeda, Chiba (JP); Masaki Tokunaga, Chiba (JP); Yuji Nagai, Chiba (JP)

(21) Appl. No.: 14/780,665

(22) PCT Filed: Apr. 2, 2014
(86) PCT No.: PCT/JP2014/060233

§ 371 Date: Sep. 28, 2015

Related U.S. Application Data
(60) Provisional application No. 61/808,419, filed on Apr. 4, 2013.

(51) Int. Cl. A61K 51/04; C07D 401/04

(52) U.S. Cl. CPC A61K 51/0459; C07D 401/04

(57) ABSTRACT

A compound selected from the group consisting of compounds represented by the formulas below or pharmaceutically acceptable salt thereof is useful in PET imaging for AMPA receptor.

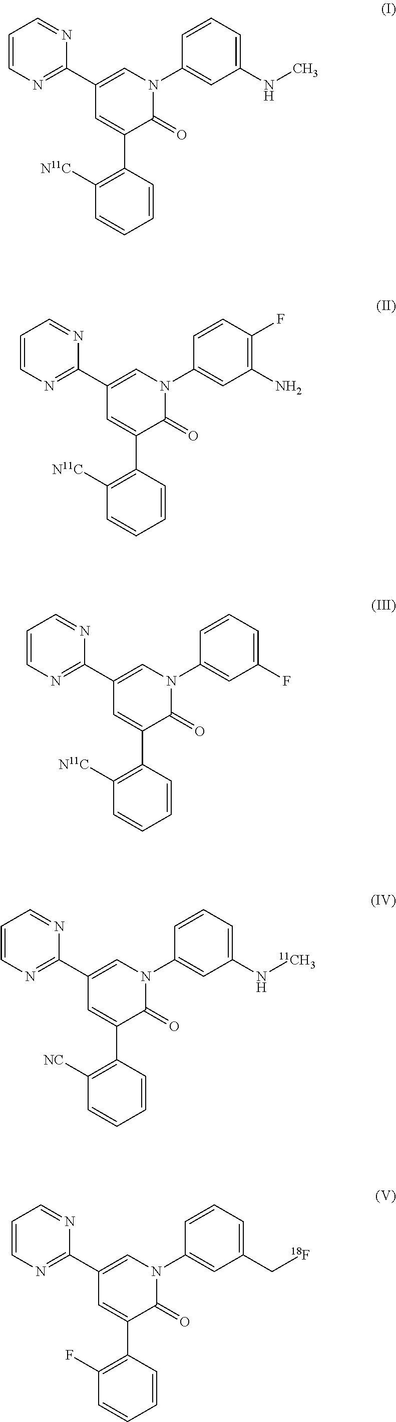

### TECHNICAL FIELD

The present invention relates to a positron emission tomography (PET) probe for AMPA receptor.

### BACKGROUND ART

The amino acid glutamate is the primary excitatory neurotransmitter in the human brain (non-patent literature 1). Glutamate exerts its physiologic effects via interaction with two major families of receptor proteins: metabotropic glutamate receptors (mGluRs) and ionotropic glutamate receptors (iGluRs). mGluRs allow glutamate to modulate cell excitability and synaptic transmission via second messenger signaling pathways, while iGluRs are ligand-gated tetrameric ion channels that mediate fast synaptic responses to glutamate. Three classes of iGluRs have been identified and are named according to their selective agonists: AMPA, kainate, and NMDA. iGluRs mediate the majority of excitatory synaptic neurotransmission in CNS (central nerve system) of higher vertebrates, and perform in the formation of synaptic plasticity underlying such as memory-leaming, differentiation-growth of nerve system in neuronal development (non-patent literature 2).

AMPA receptor acts rapid excitatory neurotransmission pathway, AMPA receptor has been highlighted on the mechanism of memory formation induced by long-term potentiation (LTP) or long term depression (LTD) (non-patent literatures 3-6). Meanwhile dysfunction of glutamatergic neurotransmission has long been implicated in the pathogenesis of neurological diseases such as epilepsy, Parkinson's disease, neuropathic pain, and stroke (non-patent literature 1). Recently, one hypothesis suggests that a mechanism involving excitotoxicity, particularly that mediated via ionotropic glutamate receptor stimulation contributes to Alzheimer's disease (AD) pathology, amyotrophic lateral sclerosis (ALS), and epilepsy (non-patent literatures 7-11).

AMPA receptors are composed of four subunits (GluR1-4) and occur as homo- or heteromultimers. Mature synapses on hippocampal pyramidal neurons are thought to contain heteromeric AMPA receptors made up of GluR1 and GluR2 and of GluR2 and GluR3. In particular, AMPA receptor channels containing the GluR2 subunit have responsibility for considerably lower Ca²⁺ permeabilities and gating behavior than receptor channels assembled without this subunit (non-patent literature 12). The Ca²⁺ permeability of the AMPA receptor is determined by Q/R editing of m-RNA, as the results that AMPA receptors keep Ca²⁺ influx in low level of neuron dominantly express GluR2/R form. The expression of the unedited GluR2/Q subunit thereby plays a critical role in determining a cell's susceptibility and increasing neurotoxicity to glutamate toxicity (non-patent literature 13).

Many AMPA receptor antagonists have been reported. Among them, perampanel has launched for the treatment of epilepsy (patent literature 1 and non-patent literature 14).

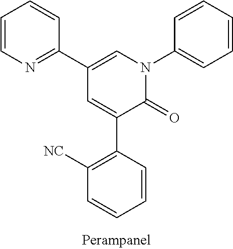 [SMILES: N#Cc1ccccc1-c1cc(-c2ccccn2)cn(-c2ccccc2)c1=O]

PET is an advanced molecular imaging modality for in vivo quantification of diverse biological processes. The relationship between AMPA receptor occupancy and dose/plasma concentration may allow us to clarify relevant dose setting and to avoid adverse events by using PET. Hence suitable PET tracers with high affinity for AMPA receptor would help to examine the relationships between the therapeutic effect and receptor occupancy of AMPA receptor antagonists. Additionally, receptor occupancy could be used as an objective outcome measure in a therapeutic assessment. Furthermore it would also help to throw light on understanding pathology which relates to AMPA receptor signaling pathway. A novel PET probe for AMPA receptor would solve biochemical events directly in living brain, and would particularly serve evaluation of drugs for diseases in which functional biomarkers are unavailable.

However only a small number of PET tracers, including N-acetyl-1-(4-chlorophenyl)-6-methoxy-7-[¹¹C]methoxy-1,2,3,4-tetrahydroisoquinoline were known (non-patent literatures 15-16). In vivo PET studies of this compound, demonstrated rapid clearance from the CNS and low specific binding in rat. Some [¹¹C] and [¹⁸F] labeled talampanel derivatives have been reported as diagnostic reagents for early diagnosis of Alzheimer's disease (patent literature 2).

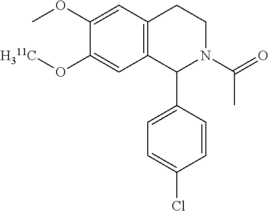 [SMILES: COc1cc2c(cc1O[11CH3])C(c1ccc(Cl)cc1)N(C(C)=O)CC2]

N-acetyl-1-(4-chlorophenyl)-6-methoxy-7-[¹¹C]methoxy-1,2,3,4-tetrahydroisoquinoline

### CITATION LIST

### Patent Literature

[Patent literature 1] WO2001/96308
[Patent literature 2] HU 1100111
[Patent literature 3] WO2009/149188

### Non Patent Literature

[Non-patent literature 1] Meldrum, B. S., Glutamate as a neurotransmitter in the brain: review of physiology and pathology. J Nutr. 2000, 130, 1007S-1015S.
[Non-patent literature 2] Asztely, F. et al., Ionotropic glutamate receptors. Their possible role in the expression of hippocampal synaptic plasticity. Mol Neurobiol. 1996, 12, 1-11.
[Non-patent literature 3] Kauer, J. A. et at, LTP: AMPA receptors trading places. Nat Neurosci. 2006, 9, 593-4.
[Non-patent literature 4] Malinow, R et al., AMPA receptor trafficking and synaptic plasticity. Annu Rev Neurosci. 2002, 25, 103-26.
[Non-patent literature 5] Chung, H. J. et al., Requirement of AMPA receptor GluR2 phosphorylation for cerebellar long-term depression. Science. 2003, 300, 1751-5.
[Non-patent literature 6] Bredt, D. S. et al., AMPA receptor trafficking at excitatory synapses. Neuron 2003, 40, 361-79.
[Non-patent literature 7] Carter, T. L. et al., Differential preservation of AMPA receptor subunits in the hippocampi of Alzheimer's disease patients according to Braak stage. Exp Neurol. 2004, 187, 299-309.
[Non-patent literature 8] Palmer, A. M. et at, Is the neuronal basis of Alzheimer's disease cholinergic or glutamatergic? FASEB J. 1990, 4, 2745-2752
[Non-patent literature 9] Sun, H. et al., Slow and selective death of spinal motor neurons in vivo by intrathecal infusion of kainic acid: implications for AMPA receptor-mediated excitotoxicity in ALS. J Neurochem. 2006, 98, 782-91.
[Non-patent literature 10] Kawahara, Y. et al., Glutamate receptors: RNA editing and death of motor neurons. Nature. 2004, 427, 801.
[Non-patent literature 11] Vollmar, W. et al., RNA editing (R/G site) and flip-flop splicing of the AMPA receptor subunit GluR2 in nervous tissue of epilepsy patients. Neurobiol Dis. 2004, 15, 371-9.
[Non-patent literature 12] Higuchi, M. et al., RNA editing of AMPA receptor subunit GluR-B: A base-paired intron-exon structure determines position and efficiency. Cell. 1993, 75, 1361-1370.
[Non-patent literature 13] Palmer, A. M. et al., Is the neuronal basis of Alzheimer's disease cholinergic or glutamatergic? FASEB J 1990, 4, 2745-2752.
[Non-patent literature 14] French, J. A. et al., Evaluation of adjunctive perampanel in patients with refractory partial-onset seizures: results of randomized global phase III study 305. Epilepsia 2013, 54, 117-25.
[Non-patent literature 15] Arstad, E et al., Closing in on the AMPA receptor: Synthesis and evaluation of 2-acetyl-1-(4′-chlorophenyl)-6-methoxy-7-[¹¹C]methoxy-1,2,3,4-tetrahydroisoquinoline as a potential PET tracer. Bioorg Med Chem Lett. 2006, 14, 4712-4717.
[Non-patent literature 16] Gao, M. et al., N-acetyl-1-aryl-6,7-dimethoxy-1,2,3,4-tetrahydroisoquinoline derivatives as new potential PET AMPA receptor ligands. Synthesis of carbon-11 and fluorine-18 labeled Bioorg Med Chem Lett. 2006, 16, 2229-2223.
[Non-patent literature 17] Iwata, R. et al., Optimization of [¹¹C]HCN production and no-carrier-added [1-¹¹C]amino acid synthesis. Int J Rad Appl Instrum A. 1987, 38, 97-102.
[Non-patent literature 18] Mathews, W. B. et al., Synthesis of a mGluR5 antagonist using [¹¹C]copper(I) cyanide. J. Labelled. Compd. Radiopharm. 2006, 49, 829-834.
[Non-patent literature 19] Solbach, C. et al., Efficient radiosynthesis of carbon-11 labelled uncharged Thioflavin T derivatives using [¹¹C]methyl triflate for beta-amyloid imaging in Alzheimer's Disease with PET. Appl Radiat Isot. 2005, 62, 591-5.
[Non-patent literature 20] Holschbach, M. et al., An on-line method for the preparation of n.c.a. [¹¹CH₃]trifluoromethanesulfonic acid methyl ester. Appl Radiat and Isot. 1993, 44, 897-898.
[Non-patent literature 21] Jewett, D. M. A simple synthesis of [¹¹C]methyl triflate. Int J Rad Appl Instrum A. 1992, 43, 1383-1385.
[Non-patent literature 22] Kawamura, K. et al., In vivo evaluation of limiting brain penetration of probes for α(2C)-adrenoceptor using small-animal positron emission tomography. ACS Chem Neurosci. 2010, 1, 520-8.
[Non-patent literature 23] Watanabe, M. et al., A high resolution animal PET scanner using compact PS-PMT detectors. IEEE Trans Nucl Sci. 1997, 44, 1277-1282.
[Non-patent literature 24] Suzuki, K. et al., Computer-controlled large scale production of high specific activity [¹¹C]RO 15-1788 for PET studies of benzodiazepine receptors. Int J Appl Radiat Isot. 1985, 36, 971-976.

### SUMMARY OF INVENTION

### Technical Problem

PET is an advanced molecular imaging modality for in vivo quantification of diverse biological processes in living brain. AMPA receptor acts rapid excitatory neurotransmission pathway, meanwhile abnormalities in AMPA signaling have been observed in various disorders such as epilepsy, ALS, and AD pathology. To visualize and identify AMPA receptor in PET imaging, an appropriate PET probe with specific binding to the target receptor is required. However, so far no PET probe which has specific affinity for AMPA receptor, especially in vivo PET imaging probe, can be available.

### Solution to Problem

To solve the problem mentioned above, we found some compounds with high affinity for AMPA receptors, and labeled these compounds. As the results, these compounds were found to be potentially useful as PET probes for AMPA receptor.

Specifically, the present invention provides the following [1] to [15].

[1] A compound selected from the group consisting of
2-(1-(3-methylaminophenyl)-2-oxo-5-(pyrimidin-2-yl)-1,2-dihydropyridin-3-yl)benzo[¹¹C]nitrile (I),
2-(1-(3-amino-4-fluorophenyl)-2-oxo-5-(pyrimidin-2-yl)-1,2-dihydropyridin-3-yl)benzo[¹¹C]nitrile (II),
2-(1-(3-fluorophenyl)-2-oxo-5-(pyrimidin-2-yl)-1,2-dihydropyridin-3-yl)benzo[¹¹C]nitile (III),
2-(1-(3-[¹¹C]methylaminophenyl)-2-oxo-5-(pyrimidin-2-yl)-1,2-dihydropyridin-3-yl)benzonitrile (IV), and
2-(1-(3-[¹⁸F]fluoromethylphenyl)-2-oxo-5-(pyrimidin-2-yl)-1,2-dihydropyridin-3-yl)benzonitrile (V)
or a pharmaceutically acceptable salt thereof.
   [2] The compound or pharmaceutically acceptable salt of [1] which is 2-(1-(3-methylaminophenyl)-2-oxo-5-(pyrimidin-2-yl)-1,2-dihydropyridin-3-yl)benzo[¹¹C]nitrile (I).
   [3] The compound or pharmaceutically acceptable salt of [1] which is 2-(1-(3-amino-4-fluorophenyl)-2-oxo-5-(pyrimidin-2-yl)-1,2-dihydropyridin-3-yl)benzo[¹¹C]nitrile (II).
   [4] The compound or pharmaceutically acceptable salt of [1] which is 2-(1-(3-fluorophenyl)-2-oxo-5-(pyrimiclin-2-yl)-1,2-dihydropyridin-3-yl)benzo[¹¹C]nitrile (III).
   [5] The compound or pharmaceutically acceptable salt of [1] which is 2-(1-(3-[¹¹C]methylaminophenyl)-2-oxo-5-(pyrimidin-2-yl)-1,2-dihydropyridin-3-yl)benzonitrile (IV).
   [6] The compound or pharmaceutically acceptable salt of [1] which is 2-(1-(3-[¹⁸F]fluoromethylphenyl)-2-oxo-5-(pyrimidin-2-yl)-1,2-dihydropyridin-3-yl)benzonitrile (V).
   [7] A composition comprising the compound or pharmaceutically acceptable salt of [1] as an active ingredient.
   [8] A PET probe comprising the compound or pharmaceutically acceptable salt of [1].
   [9] The PET probe of [8] for imaging AMPA receptor.
   [10] A method of imaging comprising administering the compound or pharmaceutically acceptable salt of [1] to a subject, and visualizing the compound or salt in the subject by PET.
   [11] The method of [10], for imaging AMPA receptor.
   [12] The compound or pharmaceutically acceptable salt of [1] for use in PET imaging.
   [13] The compound or pharmaceutically acceptable salt of [12] for imaging AMPA receptor.
   [14] Use of the compound or pharmaceutically acceptable salt of [1] for preparing a PET probe.
   [15] Use of [14] wherein the PET probe is for imaging AMPA receptor.

### Advantageous Effects of Invention

The present invention can provide a PET probes showing high Blood-brain barrier (BBB) permeability and high specific binding to AMPA receptor.

### BRIEF DESCRIPTION OF DRAWINGS

FIG. 1 is representative in vitro autoradiographic images of rat brains treated with (A) the compound of Example 4 (4.8 nM), (B) the compound of Example 4 (4.8 nM) and its unlabeled compound (Reference Example 9, 10 μM), (C) the compound of Example 2 (3.4 nM), (D) the compound of Example 2 (3.4 nM) and its unlabeled compound (Reference Example 19, 10 μM), (E) the compound of Example 3 (13.4 nM), (F) the compound of Example 3 (13.4 nM) and its unlabeled compound (Reference Example 15, 10 μM), (G) the compound of Example 5 (3.4 nM), and (H) the compound of Example 5 (3.4 nM) and its unlabeled compound (Reference Example 22, 10 μM). All sagittal slices were collected about −4 mm from bregma. HIP means hippocampus, and CTX means neocortex.

FIG. 2 is representative in vitro autoradiographic images of monkey brains. treated with (A) the compound of Example 4 (4.8 nM), (B) the compound of Example 4 (4.8 nM) and its unlabeled compound (Reference Example 9, 10 μM), (C) the compound of Example 2 (2.0 nM), (D) the compound of Example 2 (2.0 nM) and its unlabeled compound (Reference Example 19, 3.5 μM), (E) the compound of Example 3 (13.4 nM), and (F) the compound of Example 3 (13.4 nM) and its unlabeled compound (Reference Example 15, 9.6 μM). All sagittal slices were collected about 15 mm from bregma.

FIG. 3. Rhesus monkey PET study of the compound of Example 4. Orthogonal PET images of rhesus monkey brain generated by averaging dynamic scan data at 0 to 90 min after intravenous injection of the compound of Example 4 (Left). Timeradioactivity curves for the compound of Example 4 in the CTX, HIP, and brain stem (BS) of a rhesus monkey at baseline (Right). Radioactivity is expressed as percentage of standardized uptake value (% SUV).

FIG. 4. Rhesus monkey PET blocking study of the compound of Example 4. In vivo area under time-radioactivity curves of to the region of interests (CTX, HIP, cerebellum (CER), thalamus (THA), striatum (STR)) in the absence (control) and blockade by pretreatment with the unlabeled compound. Radioactivity is expressed as percentage of standardized uptake value (% SUV) and integrated radioactivity of the specific binding versus reference region (brain stem; BS) from 0 to 40 min during the PET scan.

### DESCRIPTION OF EMBODIMENTS

The compounds of the present invention are represented by the following formulas.

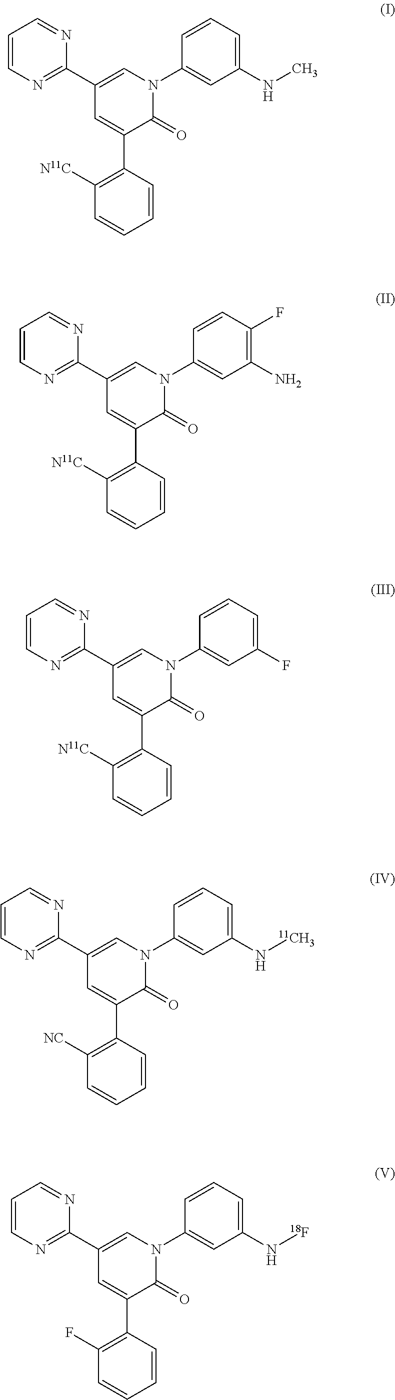

These compounds or pharmaceutically acceptable salts thereof show high Blood-brain barrier (BBB) permeability and high specific binding to AMPA receptor and thus potentially useful as PET probes for imaging AMPA receptor.

The compounds or pharmaceutically acceptable salts thereof are administered, preferably intravenously administered to a subject, and visualizing the compound or salt in the subject by PET for imaging AMPA receptor. The subject may be mammal such as human, monkey, dog, cat, rat and mouse. The subject is preferably human. A dosage of the compounds or pharmaceutically acceptable salts thereof will generally be preferably 3.1 MBq to 6.2 MBq per kg body weight.

These compounds or pharmaceutically acceptable salts thereof are prepared from the following precursors immediately before administering to a subject. Therefore, the following precursors (VI)˜(X) or salts thereof are potentially useful to prepare PET probes.

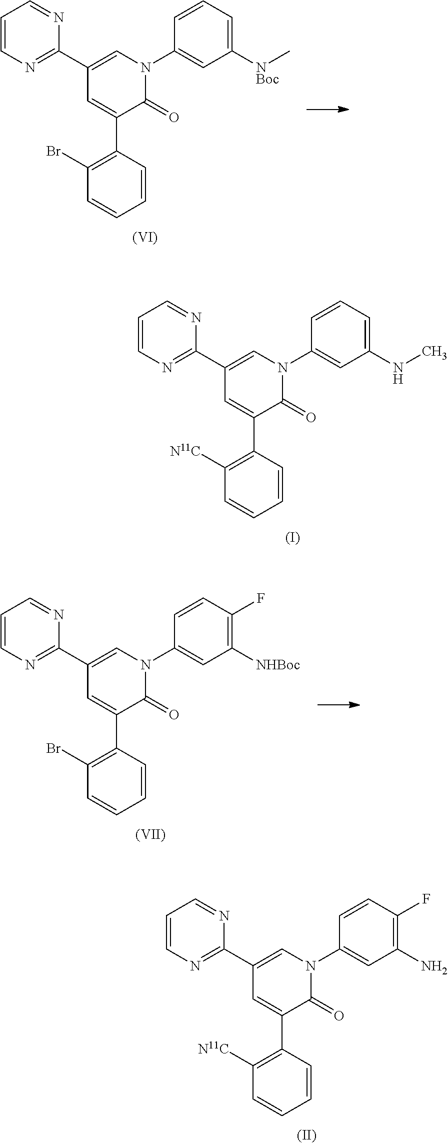

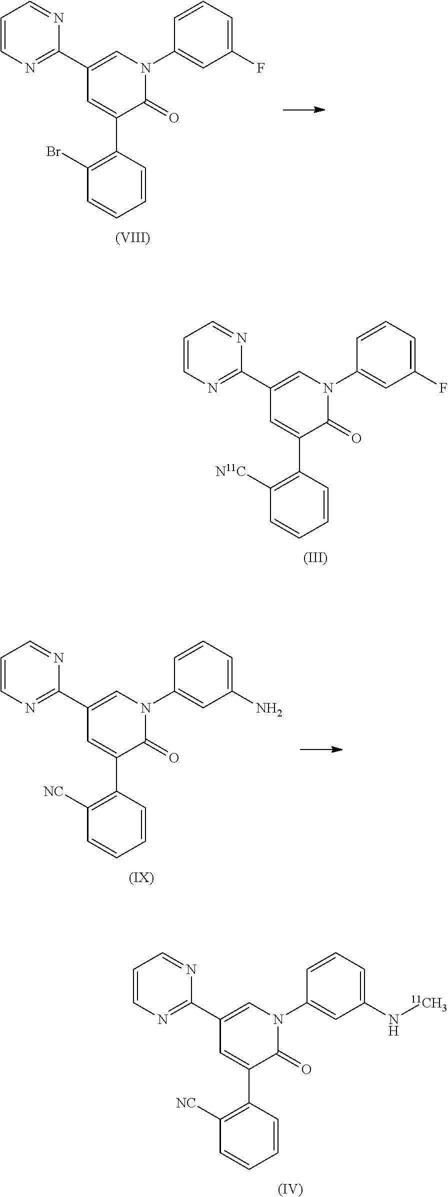

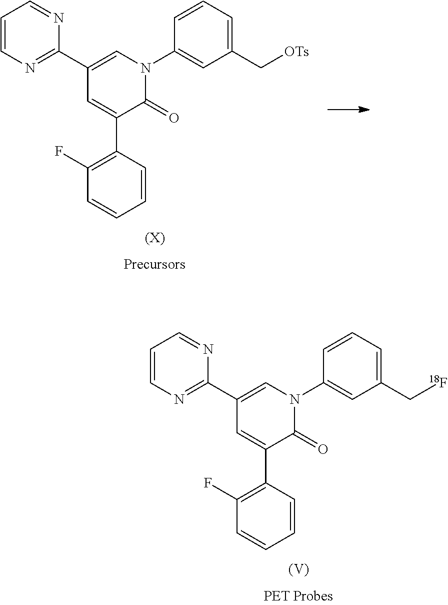

Boc means a tert-butoxycarbonyl group and Ts means a p-toluenesulfonyl group. These precursors and PET probes can be prepared by methods shown in Examples.

A “pharmaceutically acceptable salt” in the present specification is not especially limited as long as a salt formed with the compound according to the present invention, and specific examples include inorganic acid salts, organic acid salts, inorganic base salts, organic base salts, and acidic or basic amino acid salts.

If only a “pharmaceutically acceptable salt” in the present specification is a salt formed in a suitable ratio unless there is any especially limiting description, the number of acid molecules per one molecule of the compound in a formed salt, although being not especially limited, is preferably about 0.1 to about 5 molecules, more preferably about 0.5 to about 2 molecules, and still more preferably about 0.5, about 1 or about 2 molecules, per one molecule of the compound.

Preferable examples of inorganic acid salts include hydrochlorides, hydrobromides, sulfates, nitrates and phosphates, and preferable examples of organic acid salts include acetates, succinates, fumarates, maleates, tartrates, citrates, lactates, stearates, benzoates, methanesulfonates, p-toluenesulfonates and benzenesulfonates.

Preferable examples of inorganic base salts include alkaline metal salts such as sodium salts and potassium salts, alkaline earth metal salts such as calcium salts and magnesium salts, aluminum salts, and ammonium salts, and preferable examples of organic base salts include diethylamine salts, diethanolamine salts, meglumine salts and N,N′-dibenzylethylenediamine salts.

Preferable examples of acidic amino acid salts include aspartates and glutamates, and preferable examples of basic amino acid salts include arginine salts, lysine salts and ornithine salts.

The compounds or pharmaceutically acceptable salts thereof of the present invention can be formulated by conventional methods, into an appropriate dosage form such as an injection for intravenous administration. The formulation of the injection for intravenous administration can be manufactured by adding a vehicle, pH adjuster, a buffer, a suspending agent, a solubilizing agent, an antioxidant, a preservative (an antiseptic), a tonicity adjusting agent or the like to the compounds or pharmaceutically acceptable salts thereof of the present invention as necessary and treating by conventional methods.

Examples of the vehicles include sterile saline, examples of the pH adjuster and buffer include organic acids or inorganic acids and/or salts thereof, examples of the suspending agent include methylcellulose, polysorbate 80 and carboxymethylcellulose sodium, examples of the solubilizing agent include polysorbate 80 and polyoxyethylene sorbitan monolaurate, examples of the antioxidant include ascorbic acid, α-tocopherol, examples of the preservative include methyl parahydroxybenzoate and ethyl parahydroxybenzoate, and examples of the tonicity adjusting agent include glucose, sodium chloride and mannitol.

### EXAMPLES

The invention will now be described in greater detail by examples, test examples, and reference examples, with the understanding that the invention is not limited to the examples.

### Reference Example 1

### Synthesis of 3-iodo-5-(pyrimidin-2-yl)pyridin-2(1H)-one

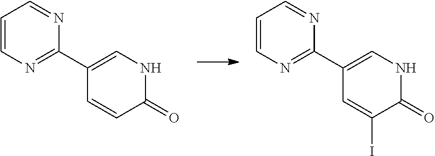 [SMILES: O=c1ccc(-c2ncccn2)c[nH]1 ; O=c1[nH]cc(-c2ncccn2)cc1I]

A mixture of 5-(pyrimidin-2-yl)pyridin-2(1H)-one (Patent literatures 1 and 3) (4.5 g, 31 mmol) and N-iodosuccinimide (7.0 g, 31 mmol) in chloroform (100 mL) was refluxed for 2 h and cooled to room temperature. The reaction mixture was diluted with dichloromethane and filtered. The precipitate was washed with diethyl ether to give the title compound (6.7 g, 86% yield).

¹H-NMR (300 MHz, DMSO-d₆, δ): 7.35 (t, J=4.6 Hz, 1H), 8.38 (d, J=1.4 Hz, 1H), 8.81 (d, J=4.6 Hz, 2H), 8.93 (d, J=1.4 Hz, 1H), 12.36 (brs, 1H).

ESI-MS: m/z 300 (M+H), 621 (2M+Na).

HRMS (FAB): calcd for C₉H₇IN₃O, 299.9634. found, 299.9636.

### Reference Example 2

### Synthesis of 3-bromo-5-(pyrimidin-2-yl)pyridin-2(1H)-one

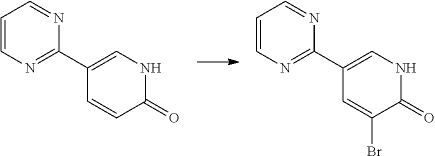 [SMILES: O=c1ccc(-c2ncccn2)c[nH]1 ; O=c1[nH]cc(-c2ncccn2)cc1Br]

A mixture of 5-(pyrimidin-2-yl)pyridin-2(1H)-one (2.4 g, 13.7 mmol) and N-bromosuccinimide (2.4 g, 13.7 mmol) in N,N-dimethylformamide (DMF) (20 mL) was stirred for 2 h at room temperature. The reaction mixture was diluted with water and filtered. The precipitate was washed with water to give the title compound (1.8 g, 51% yield).

¹H-NMR (300 MHz, DMSO-d₆, δ): 7.35 (t, J=4.8 Hz, 1H), 8.36 (s, 1H), 8.67 (s, 1H), 8.80 (d, J=4.8 Hz, 2H), 12.54 (brs, 1H).

ESI-MS: m/z 252 (M+H), 525 (2M+Na).

HRMS (FAB): calcd for C₉H₇BrN₃O, 251.9772. found, 251.9737.

### Reference Example 3

### Synthesis of tert-butyl N-(3-(3-iodo-2-oxo-5-(pyrimidin-2-yl)-1,2-dihydropyridin-1-yl)phenyl)carbamate

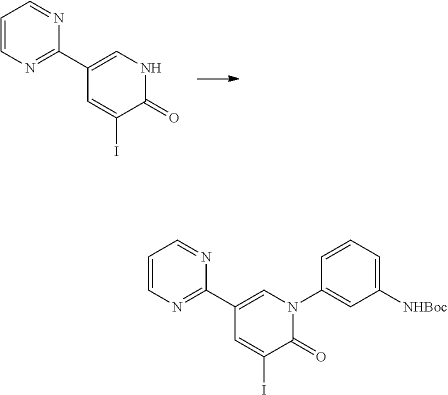 [SMILES: O=c1[nH]cc(-c2ncccn2)cc1I ; CC(C)(C)OC(=O)Nc1cccc(-n2cc(-c3ncccn3)cc(I)c2=O)c1]

A mixture of the compound of Reference Example 1 (750 mg, 2.5 mmol), N-tert butoxycarbonyl-3-aminophenylboronic acid (1.5 g, 6.3 mmol), copper(II) acetate (1.37 g, 7.5 mmol) and triethylamine (4.3 mL, 31 mmol) in tetrahydrofuran (THF) (50 mL) was stirred at 60° C. for 2 h. The reaction mixture was diluted with an aqueous ammonia solution and insoluble material was filtered off. The filtrate was extracted with ethyl acetate and dried over anhydrous magnesium sulfate. The organic layer was evaporated in vacuo and the residue was purified by silica gel column chromatography using n-heptane/ethyl acetate (3/7, v/v) to give the title compound (470 mg, 38% yield).

¹H-NMR (300 MHz, CDCl₃, δ): 1.51 (s, 9H), 6.66 (brs, 1H), 7.09 (brd, J=8.0 Hz, 1H), 7.15 (t, J=4.9 Hz, 1H), 7.31 (brd, J=8.0 Hz, 1H), 7.40 (t, J=8.0 Hz, 1H), 7.64 (brs, 1H), 8.63 (d, J=2.3 Hz, 1H), 8.69 (3, J=4.9 Hz, 2H), 9.12 (d, J=2.3 Hz, 1H).

ESI-MS: m/z 491 (M+H).

HRMS (FAB) calcd for C₂₀H₂₀IN₄O₃, 491.0580. found, 491.0554.

### Reference Example 4

### Synthesis of tert-butyl N-(3-(3-(2-cyanophenyl)-2-oxo-5-(pyrimidin-2-yl)-1,2-dihydropyridin-1-yl)phenyl)carbamate

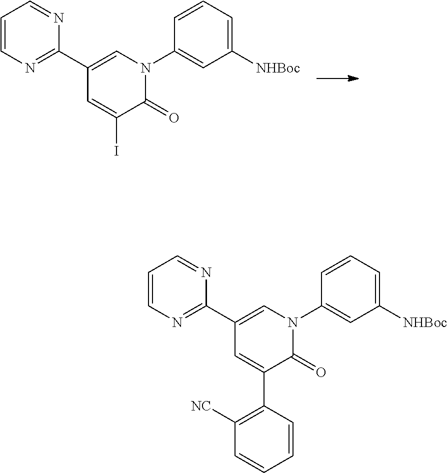 [SMILES: CC(C)(C)OC(=O)Nc1cccc(-n2cc(-c3ncccn3)cc(I)c2=O)c1 ; CC(C)(C)OC(=O)Nc1cccc(-n2cc(-c3ncccn3)cc(-c3ccccc3C#N)c2=O)c1]

A mixture of the compound of Reference Example 3 (225 mg, 0.45 mmol), 2-(1,3,2-dioxaboran-2-yl)benzonitrile (258 mg, 1.35 mmol), cesium carbonate (220 mg, 0.68 mmol) and tetrakis(triphenylphosphine)palladium(0) (52 mg, 0.03 mmol) in DMF (10 mL) was stirred at 110° C. for 2 h under nitrogen atmosphere. The reaction mixture was diluted with water, was extracted with ethyl acetate and dried over anhydrous magnesium sulfate. The organic layer was evaporated in vacuo and the residue was purified by silica gel column chromatography using n-heptane/ethyl acetate (5/5, v/v) to give the title compound (200 mg, 90% yield).

¹H-NMR. (400 MHz, CDCl₃, δ): 1.51 (s, 9H), 6.62 (brs, 1H), 7.14 (t, J=4.8 Hz, 1H), 7.13-7.20 (m, 1H), 7.37-7.48 (m, 3H), 7.60-7.68 (m, 2H), 7.72 (d, J=7.9 Hz, 1H), 7.76 (d, J=7.9 Hz, 1H), 8.70 (d, J=4.8 Hz, 2H), 8.71 (d, J=2.6 Hz, 1H), 8.74 (d, J=2.6 Hz, 1H).

ESI-MS: m/z 466 (M+H).

HRMS (FAB) calcd for C₂₇H₂₄N₅O₃, 466.1879. found, 466.1842.

### Reference Example 5

### Synthesis of tert-butyl N-(3-(3-(2-cyanophenyl)-2-oxo-5-(pyrimidin-2-yl)-1,2-dihydropyridin-1-yl)phenyl)(methyl)carbamate

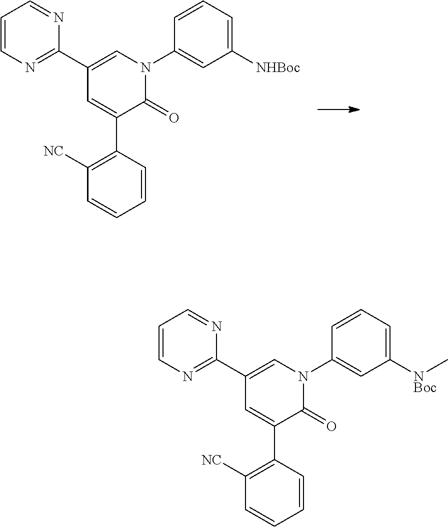 [SMILES: CC(C)(C)OC(=O)Nc1cccc(-n2cc(-c3ncccn3)cc(-c3ccccc3C#N)c2=O)c1 ; CN(C(=O)OC(C)(C)C)c1cccc(-n2cc(-c3ncccn3)cc(-c3ccccc3C#N)c2=O)c1]

Sodium hydride (30 mg, 60% in oil, 0.75 mmol) was added to a solution of the compound of Reference Example 4 (200 mg, 0.43 mmol) in DMF (5 mL) at 0° C., and stirred at room temperature for 5 min. Iodomethane (40 μL, 0.64 mmol) was added to the reaction mixture and stirred for 10 min. The reaction mixture was quenched with brine and extracted with ethyl acetate. The organic layer was dried over anhydrous magnesium sulfate and evaporated in vacuo. The residue was purified by silica gel column chromatography using n-heptane/ethyl acetate (7/3, v/v) to give the title compound (185 mg, 90% yield).

¹H-NMR (300 MHz, CDCl₃, δ): 1.42 (s, 9H), 3.17 (s, 3H), 7.08 (t, J=4.8 Hz, 1H), 7.22-7.27 (m, 1H), 7.27-7.33 (m, 1H), 7.34-7.44 (m, 3H), 7.53-7.61 (m, 1H), 7.64 (d, J=8.0 Hz, 1H), 7.70 (d, J=8.0 Hz, 1H), 8.60-8.66 (m, 3H), 8.69 (d, J=2.4 Hz, 1H).

ESI-MS: m/z 502 (M+Na).

HRMS (FAB) calcd for C₂₈H₂₆N₅O₃, 480.2036. found, 480.2044.

### Reference Example 6

### Synthesis of 2-(1-(3-aminophenyl)-2-oxo-5-(pyrimidin-2-yl)-1,2-dihydropyridin-3-yl)benzonitrile

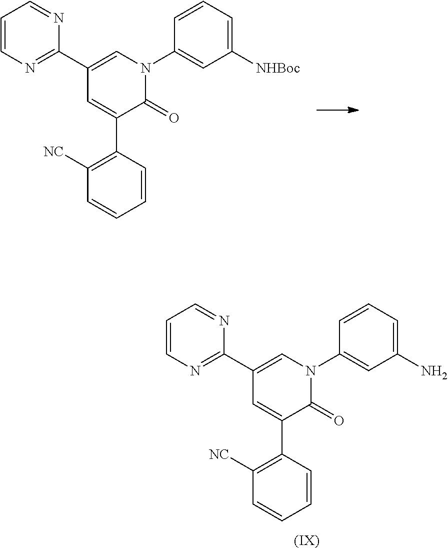

The compound of Reference Example 4 (50 mg, 0.11 mmol) was dissolved in trifluoroacetic acid (2 mL) and the mixture was stirred for 15 min at room temperature. The excess amount of trifluoroacetic acid was removed in vacuo, the residue was neutralized with a saturated aqueous solution of sodium hydrogen carbonate, extracted with ethyl acetate, and the extract was dried over anhydrous magnesium sulfate. The organic layer was evaporated in vacuo and the residue was purified by silica gel column chromatography using n-heptane/ethyl acetate (5/5 to 0/1, v/v) to give the title compound (30 mg, 77% yield).

¹H-NMR (300 MHz, CDCl₃, δ): 3.85 (brs, 2H), 6.76 (d, J=9.0 Hz, 1H), 6.80-6.90 (m, 2H), 7.14 (t, J=5.5 Hz, 1H), 7.26-7.33 (m, 1H), 7.45 (t, J=8.1 Hz, 1H), 7.63 (t, J=8.1 Hz, 1H), 7.69-7.80 (m, 2H), 8.65-8.73 (m, 3H), 8.75 (d, J=2.6 Hz, 1H).

¹³C-NMR (150 MHz, DMSO-d₆, δ): 111.5, 111.9, 113.2, 114.0, 115.5, 118.1, 119.3, 128.6, 128.6, 129.6, 130.8, 132.9, 133.0, 138.0, 140.3, 140.6, 141.6, 149.7, 157.7 2*C, 159.6, 160.6.

HRMS (FAB) calcd for C₂₂H₁₆N₅O, 366.1355. found, 366.1384.

### Reference Example 7

### Synthesis of tert-butyl N-(3-(3-(2-bromophenyl)-2-oxo-5-(pyrimidin-2-yl)-1,2-dihydropyridin-1-yl)phenyl)carbamate

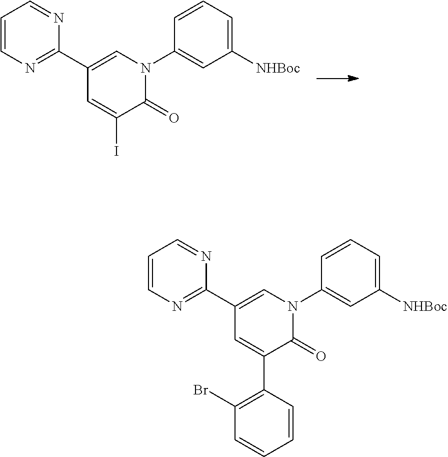 [SMILES: CC(C)(C)OC(=O)Nc1cccc(-n2cc(-c3ncccn3)cc(I)c2=O)c1 ; CC(C)(C)OC(=O)Nc1cccc(-n2cc(-c3ncccn3)cc(-c3ccccc3Br)c2=O)c1]

A mixture of the compound of Reference Example 3 (150 mg, 0.31 mmol), 2-bromophenylboronic acid (184 mg, 0.92 mmol), cesium carbonate (250 mg, 0.77 mmol) and tetrakis(triphenylphosphine)palladium(0) (35 mg, 0.03 mmol) in DMF (5 mL) was stirred at 110° C. for 2 h under nitrogen atmosphere. The reaction mixture was diluted with water, extracted with ethyl acetate and dried over anhydrous magnesium sulfate. The organic layer was evaporated in vacuo and the residue was purified by silica gel column chromatography using n-heptane/ethyl acetate (5/5, v/v) to give the title compound (140 mg, 88% yield).

¹H-NMR (400 MHz, CDCl₃, δ): 1.52 (s, 9H), 6.60 (brs, 1H), 7.13 (t, J=4.7 Hz, 1H), 7.16-7.24 (m, 2H), 7.33-7.38 (m, 2H), 7.38-7.47 (m, 2H), 7.64-7.69 (m, 2H), 8.50 (d, J=2.4 Hz, 1H), 8.69 (d, J=4.7 Hz, 2H), 8.72 (d, J=2.4 Hz, 1H).

ESI-MS: m/z 519 (M+H).

HRMS (FAB) calcd for C₂₆H₂₄BrN₄O₃, 519.1032. found, 519.1014.

### Reference Example 8

### Synthesis of tert-butyl (3-(3-(2-bromophenyl)-2-oxo-5-(pyrimidin-2-yl)pyridin-1(2H)-yl)phenyl)(methyl)carbamate

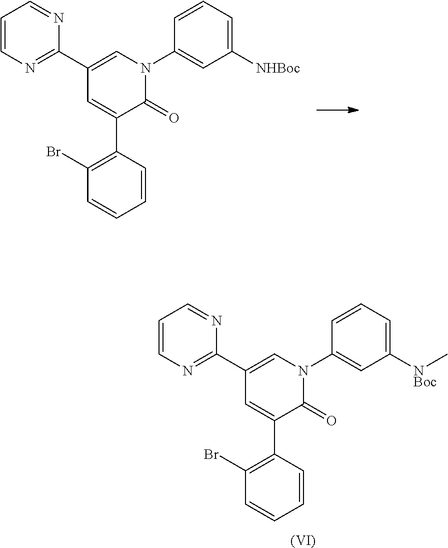

Sodium hydride (9 mg, 60% in oil, 0.23 mmol) was added to a solution of the compound of Reference Example 7 (60 mg, 0.12 mmol) in DMF (2 mL) at 0° C., and stirred at room temperature for 5 min. Iodomethane (12 μL, 0.64 mmol) was added to the reaction mixture and stirred for 10 min. The reaction mixture was quenched with brine and extracted with ethyl acetate. The organic layer was dried over anhydrous magnesium sulfate and evaporated in vacuo. The residue was purified by silica gel column chromatography using n-heptane/ethyl acetate (7/3, v/v) to give the title compound (45 mg, 73% yield).

¹H-NMR (400 MHz, CDCl₃, δ): 1.49 (s, 9H), 3.30 (s, 3H), 7.14 (t, J=5.0 Hz, 1H), 7.20-7.25 (m, 1H), 7.28-7.39 (m, 3H), 7.42-7.48 (m, 3H), 7.67 (d, J=8.2 Hz, 1H), 8.51 (d, J=2.7 Hz, 1H), 8.69 (d, J=4.8 Hz, 2H), 8.74 (d, J=2.7 Hz, 1H).

ESI-MS: m/z 533 (M+H).

HRMS (FAB) calcd for C₂₇H₂₆BrN₄O₃, 533.1188. found, 533.1166.

### Reference Example 9

### Synthesis of 2-(1-(3-(methylamino)phenyl)-2-oxo-5-(pyrimidin-2-yl)-1,2-dihydropyridin-3-yl)benzonitrile

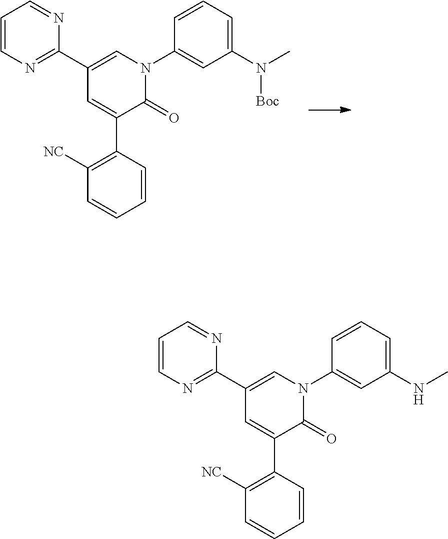 [SMILES: CN(C(=O)OC(C)(C)C)c1cccc(-n2cc(-c3ncccn3)cc(-c3ccccc3C#N)c2=O)c1 ; CNc1cccc(-n2cc(-c3ncccn3)cc(-c3ccccc3C#N)c2=O)c1]

The compound of Reference Example 5 (180 mg, 0.38 mmol) was dissolved in trifluoroacetic acid (2 mL) at 0° C. and the mixture was stirred for 10 min. The excess amount of trifluoroacetic acid was removed in vacuo, the residue was neutralized with a saturated aqueous solution of sodium hydrogen carbonate, extracted with ethyl acetate, and the extract was dried over anhydrous magnesium sulfate. The organic layer was evaporated in vacuo and crystallized in diisopropyl ether to give the title compound (120 mg, 85% yield).

¹H-NMR (400 MHz, CDCl₃, δ): 2.87 (brs, 3H), 3.94 (brs, 1H), 6.66-6.71 (m, 1H), 6.72 (t, J=2.1 Hz, 1H), 6.80 (ddd, J=7.9, 2.1, 0.9 Hz, 1H), 7.14 (t, J=4.8 Hz, 1H), 7.31 (t, J=7.9 Hz, 1H), 7.42-7.48 (m, 1H), 7.61-7.67 (m, 1H), 7.73-7.79 (m, 2H), 8.70-8.72 (m, 3H), 8.77 (d, J=2.6 Hz, 1H).

¹³C-NMR (150 MHz, DMSO-d₆, δ): 29.6, 109.2, 111.9, 112.1, 113.1, 115.5, 118.1, 119.3, 128.5, 128.7, 129.6, 130.8, 133.0, 133.0, 138.1, 140.3, 140.7, 141.8, 150.8, 157.7 2*C, 159.7, 160.6.

ESI-MS: m/z 380 (M+H).

HRMS (FAB) calcd for C₂₃H₁₈N₅O, 380.1511. found, 380.1517.

### Reference Example 10

### Synthesis of 3-iodo-1-(3-dimethylaminophenyl)-5-(pyrimidin-2-yl)pyridin-2(1H)-one

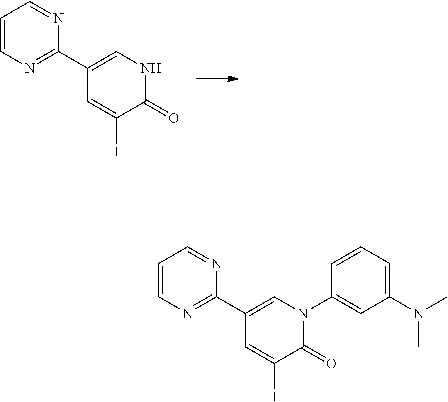 [SMILES: O=c1[nH]cc(-c2ncccn2)cc1I ; CN(C)c1cccc(-n2cc(-c3ncccn3)cc(I)c2=O)c1]

A mixture of the compound of Reference Example 1 (590 mg, 2.0 mmol), 3-dimethylaminophenylboronic acid (990 mg, 6.0 mmol), copper(II) acetate (1.09 g, 6.0 mmol) and triethylamine (3.3 mL, 24 mmol) in chloroform (50 mL) was stirred at room temperature for 8 h. The reaction mixture was diluted with an aqueous ammonia solution, the resulting solution was extracted with ethyl acetate and dried over anhydrous magnesium sulfate. The organic layer was evaporated in vacuo and the residue was purified by silica gel column chromatography using ethyl acetate/dichloromethane (1/4, v/v) and was crystallized in diisopropylether to give the title compound (650 mg, 79% yield).

¹H-NMR (300 MHz, CDCl₃, δ): 2.98 (s, 6H), 6.67-6.73 (m, 2H), 6.78 (dd, J=3.1, 8.4 Hz, 1H), 7.13 (t, J=4.7 Hz, 1H), 7.33 (t, J=8.4 Hz, 1H), 8.66 (d, J=2.2 Hz, 1H), 8.69 (d, J=4.7 Hz, 2H), 9.12 (d, J=2.2 Hz, 1H).

HRMS (FAB) calcd for C₁₇H₁₆IN₄O, 419.0369. found, 419.0373.

### Reference Example 11

### Synthesis of 2-(1-(3-dimethyaminophenyl)-2-oxo-5-(pyrimidin-2-yl)-1,2-dihydropyridin-3-yl)benzonitrile

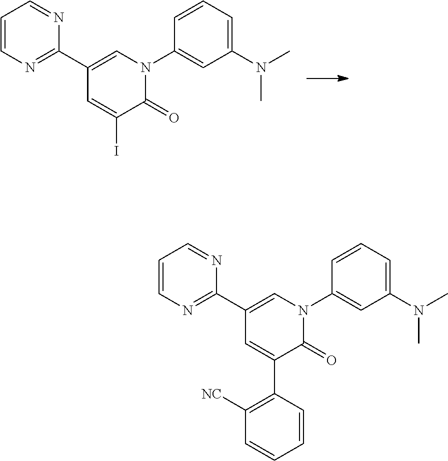 [SMILES: CN(C)c1cccc(-n2cc(-c3ncccn3)cc(I)c2=O)c1 ; CN(C)c1cccc(-n2cc(-c3ncccn3)cc(-c3ccccc3C#N)c2=O)c1]

A mixture of the compound of Reference Example 10 (414 mg, 1.0 mmol), 2-(1,3,2-dioxaboran-2-yl)benzonitrile (560 mg, 3.0 mmol), cesium carbonate (490 mg, 1.5 mmol) and tetrakis(triphenylphosphine)palladium(0) (120 mg, 0.10 mmol) in DMF (10 mL) was stirred at 115° C. for 20 min under nitrogen atmosphere. The reaction mixture was diluted with water, was extracted with ethyl acetate and dried over anhydrous magnesium sulfate. The organic layer was evaporated in vacuo and the residue was purified by silica gel column chromatography using n-heptane/ethyl acetate (4/6, v/v) to give the title compound (350 mg, 89% yield).

¹H-NMR (300 MHz, CDCl₃, δ): 2.99 (s, 6H), 6.77-6.83 (m, 3H), 7.13 (t, J=4.8 Hz, 1H), 7.33 (t, J=8.3 Hz, 1H), 7.44 (t, J=7.7 Hz, 1H), 7.62 (t, J=7.7 Hz, 1H), 7.73-7.79 (m, 2H), 8.70 (d, J=4.8 Hz, 2H), 8.71 (d, J=2.6 Hz, 1H), 8.77 (d, J=2.6 Hz, 1H).

¹³C-NMR (150 MHz, DMSO-d₆, δ): 40.0 2*C, 110.2, 111.9, 112.4, 113.7, 115.5, 118.2, 119.3, 128.5, 128.4, 128.6, 130.8, 133.0, 133.0, 138.1, 140.3, 140.8, 141.8, 151.1, 157.7 2*C, 159.8, 160.7.

HRMS (FAB) calcd for C₂₄H₂₀IN₅O, 394.1668. found, 394.1698.

### Reference Example 12

### Synthesis of 3-iodo-1-(3-fluorophenyl)-5-(pyrimidin-2-yl)pyridin-2(1H)-one

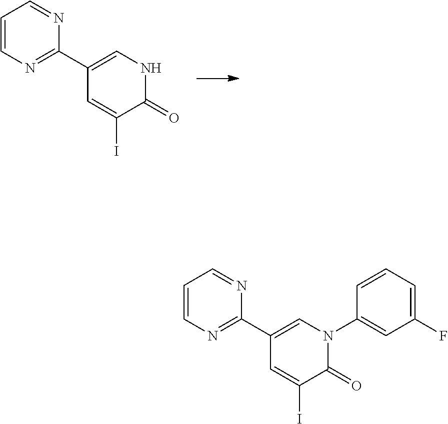 [SMILES: O=c1[nH]cc(-c2ncccn2)cc1I ; O=c1c(I)cc(-c2ncccn2)cn1-c1cccc(F)c1]

A mixture of the compound of Reference Example 1 (1.0 g, 3.4 mmol), 3-fluorophenylboronic acid (1.4 g, 10.2 mmol), copper(II) acetate (1.85 g, 10.2 mmol) and triethylamine (5.6 mL, 40.8 mmol) in chloroform (50 mL) was stirred at room temperature for 12 h. The reaction mixture was diluted with an aqueous ammonia solution and extracted with ethyl acetate and dried over anhydrous magnesium sulfate. The organic layer was evaporated in vacuo and the residue was purified by silica gel column chromatography using ethyl acetate/chloroform (2/8, v/v) to give the title compound (1.3 g, 98% yield).

¹H-NMR (300 MHz, CDCl₃, δ): 7.14-7.28 (m, 3H), 7.17 (t, J=5.1 Hz, 1H), 7.50 (dt, J=5.6, 8.1 Hz, 1H), 8.62 (d, J=2.2 Hz, 1H), 8.71 (d, J=5.1 Hz, 2H), 9.15 (d, J=2.2 Hz, 1H).

HRMS (FAB) calcd for C₁₅H₁₀FIN₃O, 393.9853. found, 393.9812.

### Reference Example 13

### Synthesis of 3-bromo-1-(3-fluorophenyl)-5-(pyrimidin-2-yl)pyridin-2(1H)-one

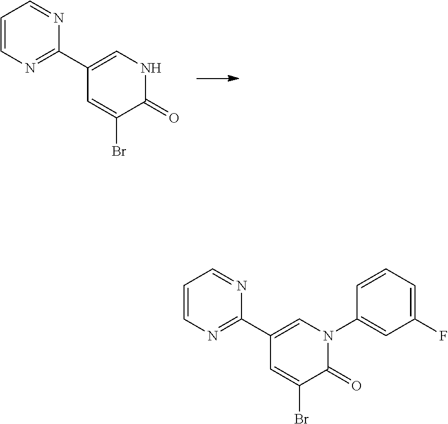 [SMILES: O=c1[nH]cc(-c2ncccn2)cc1Br ; O=c1c(Br)cc(-c2ncccn2)cn1-c1cccc(F)c1]

A mixture of the compound of Reference Example 2 (200 mg, 0.79 mmol), 3-fluorophenylboronic acid (333 mg, 2.38 mmol), copper(II) acetate (1.85 g, 10.2 mmol) and pyridine (642 μL, 7.93 mmol) in DMF (30 mL) and chloroform (50 mL) was stirred at 60° C. for 8 h. The reaction mixture was diluted with a 10% aqueous ammonia solution and stirred for 30 min. The resulting solid was filtered, rinsed with water, and collected. The solid was dried in vacuo to give the title compound (199 mg, 73% yield).

¹H-NMR (400 MHz, CDCl₃, δ): 7.17 (t, J=5.0 Hz, 1H), 7.21-7.27 (m, 3H), 7.47-7.55 (m, 1H), 8.61 (d, J=2.4 Hz, 1H), 8.71 (d, J=4.7 Hz, 2H), 8.91 (d, J=2.4 Hz, 1H).

ESI-MS: m/z 346 (M+H).

### Reference Example 14

### Synthesis of 3-(2-bromophenyl)-1-(3-fluorophenyl)-5-(pyrimidin-2-yl)pyridin-2(1H)-one

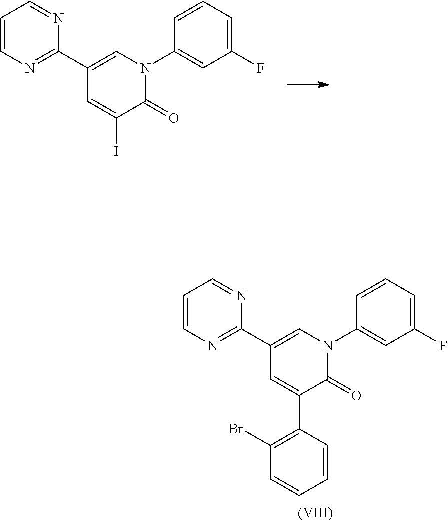

A mixture of the compound of Reference Example 12 (1.30 g, 3.34 mmol), 2-bromophenylboronic acid (1.34 g, 6.68 mmol), cesium carbonate (1.63 g, 5.00 mmol) and tetrakis(triphenylphosphine)palladium(0) (190 mg, 0.17 mmol) in DMF (20 mL) was stirred at 115° C. for 45 min under nitrogen atmosphere. The reaction mixture was diluted with water, was extracted with ethyl acetate and dried over anhydrous magnesium sulfate. The organic layer was evaporated in vacuo and the residue was purified by silica gel column chromatography using n-heptane/ethyl acetate (2/1, v/v) and crystallized in diisopropyl ether to give the title compound (820 mg, 67% yield).

¹H-NMR (400 MHz, CDCl₃, δ): 7.14-7.20 (m, 1H), 7.15 (d, J=4.9 Hz, 1H), 7.20-7.26 (m, 1H), 7.29-7.40 (m, 3H), 7.42-7.53 (m, 2H), 7.67 (dd, J=8.1, 1.0 Hz, 1H), 8.52 (d, J=2.4 Hz, 1H), 8.67-8.74 (m, 3H).

ESI-MS: m/z 422 (M+H).

HRMS (FAB) calcd for C₂₁H₁₄BrFN₃O, 422.0304. found, 422.0255.

### Reference Example 15

### Synthesis of 2-(1-(3-fluorophenyl)-2-oxo-5-(pyrimidin-2-yl)-1,2-dihydropyridin-3-yl)benzonitrile

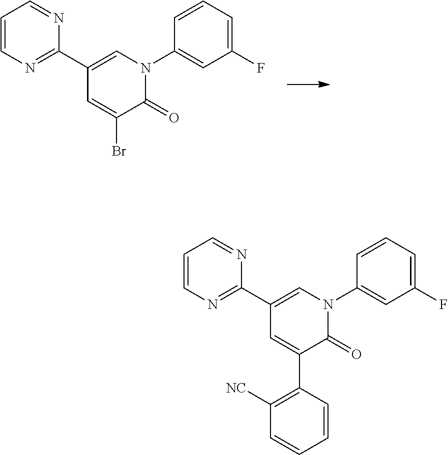 [SMILES: O=c1c(Br)cc(-c2ncccn2)cn1-c1cccc(F)c1 ; N#Cc1ccccc1-c1cc(-c2ncccn2)cn(-c2cccc(F)c2)c1=O]

A mixture of the compound of Reference Example 13 (30 mg, 0.09 mmol), 2-0,3,2-dioxaboran-2-yl)benzonitrile (32 mg, 0.17 mmol), cesium carbonate (85 mg, 0.26 mmol) and tetrakis(triphenylphosphine)palladium(0) (5 mg, 4.3 μmol) in DMF (4 mL) was stirred at 120° C. for 3 h under nitrogen atmosphere. The reaction mixture was diluted with water, was extracted with ethyl acetate and dried over anhydrous magnesium sulfate. The organic layer was evaporated in vacuo and the residue was purified by silica gel column chromatography using n-heptane/ethyl acetate (1/4, v/v) to give the title compound (19 mg, 60% yield).

¹H-NMR (400 MHz, CDCl₃, δ): 7.16-7.23 (m, 1H), 7.17 (t, J=4.7 Hz, 1H), 7.30-7.38 (m, 2H), 7.45-7.56 (m, 2H), 7.66 (t, J=7.9 Hz, 1H), 7.72 (d, J=7.9 Hz, 1H), 7.78 (d, J=7.9 Hz, 1H), 8.72 (d, J=4.7 Hz, 2H), 8.73 (d, J=2.7 Hz, 1H), 8.74 (d, J=2.7 Hz, 1H).

ESI-MS: m/z 369 (M+H).

HRMS (FAB) calcd for C₂₂H₁₄FN₄O, 369.1152. found, 369.1106.

### Reference Example 16

### Synthesis of tert-butyl (2-fluoro-5-(3-iodo-2-oxo-5-(pyrimidin-2-yl)pyridin-1(2H)-yl)phenyl)carbamate

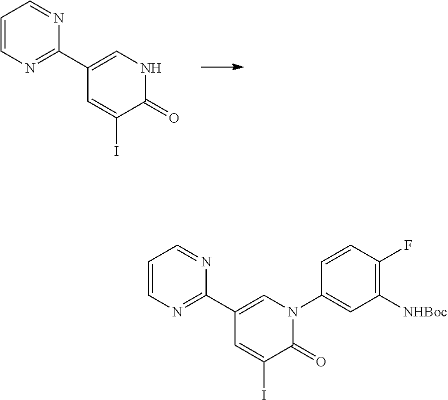 [SMILES: O=c1[nH]cc(-c2ncccn2)cc1I ; CC(C)(C)OC(=O)Nc1cc(-n2cc(-c3ncccn3)cc(I)c2=O)ccc1F]

A solution of triethylamine (460 μL, 3.34 mmol) and catalytic amount of N,N-dimethylaminopyridine was added to a solution of di-tert-butyl dicarbonate (730 mg, 3.34 mmol) and 3-amino-4-fluorophenylboronic acid (520 mg, 3.34 mmol) in THF (20 mL) and stirred at room temperature for 12 h. The reaction mixture was acidified with HCl, subsequently extracted with ethyl acetate. The extract was dried over anhydrous magnesium sulfate and evaporated in vacuo. The residue was dissolved in chloroform (10 mL), the resulting solution was added the compound of Reference Example 1 (500 mg, 1.67 mmol), copper(II) acetate (911 mg, 5.0 mmol) and triethylamine (2.8 mL, 20.1 mmol) was stirred at 50° C. for 1 h. The reaction mixture was diluted with an aqueous ammonia solution and extracted with ethyl acetate and dried over anhydrous magnesium sulfate. The organic layer was evaporated in vacuo and the residue was purified by silica gel column chromatography using n-heptane/ethyl acetate (1/1, v/v) to give the title compound (400 mg, 47% yield).

¹H-NMR (400 MHz, CDCl₃, δ): 1.53 (s, 9H), 6.83 (brs, 1H), 7.04 (ddd, J=2.6, 5.4, 8.6 Hz, 1H), 7.15 (t, J=4.8 Hz, 1H), 7.18 (dd, J=8.6, 10.4 Hz, 1H), 8.27 (brd, J=5.4 Hz, 1H), 8.60 (d, J=2.4 Hz, 1H), 8.69 (d, J=4.8 Hz, 2H), 9.12 (d, J=2.4 Hz, 1H).

ESI-MS: m/z 509 (M+H).

HRMS (FAB) calcd for C₂₀H₁₉IN₄O₃, 509.0486. found, 509.0523.

### Reference Example 17

### Synthesis of tert-butyl (5-(3-(2-bromophenyl)-2-oxo-5-(pyrimidin-2-yl)pyridin-1(2H)-yl)-2-fluorophenyl)carbamate

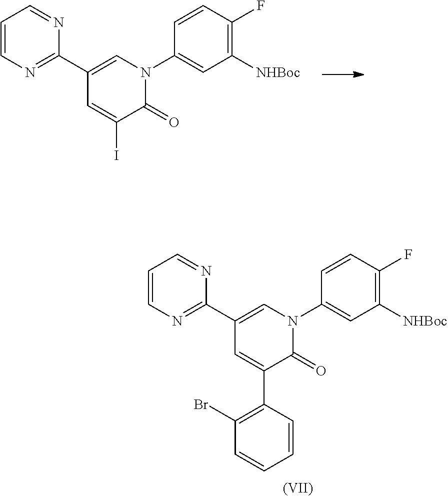

A mixture of the compound of Reference Example 16 (130 mg, 0.26 mmol), 2-bromophenylboronic acid (128 mg, 0.64 mmol), cesium carbonate (210 mg, 0.64 mmol) and tetrakis(triphenylphosphine)palladium(0) (30 mg, 26 μmol) in DMF (10 mL) was stirred at 120° C. for 1 h under nitrogen atmosphere. The reaction mixture was diluted with water, extracted with ethyl acetate and dried over anhydrous magnesium sulfate. The organic layer was evaporated in vacuo and the residue was purified by silica gel column chromatography using n-heptane/ethyl acetate (1/4, v/v) to give the title compound (100 mg, 73% yield).

¹H-NMR (400 MHz, CDCl₃, δ): 1.52 (s, 9H), 6.82 (brs, 1H), 7.10-7.17 (m, 314), 7.21 (t, J=7.8 Hz, 1H), 7.37 (t, J=7.8 Hz, 1H), 7.44 (d, J=7.8 Hz, 1H), 7.65 (d, J=7.8 Hz, 1H), 8.35 (brd, J=5.0 Hz, 1H), 8.51 (d, J=2.4 Hz, 1H), 8.67-8.71 (m, 1H), 8.69 (d, J=4.8 Hz, 2H). ESI-MS: m/z 537 (M+H).

HRMS (FAB) calcd for C₂₆H₂₃BrFN₄O₃, 537.0958. found, 537.0938.

### Reference Example 18

### Synthesis of tert-butyl (5-(3-(2-cyanophenyl)-2-oxo-5-(pyrimidin-2-yl)pyridin-1(2H)-yl)-2-fluorophenyl)carbamate

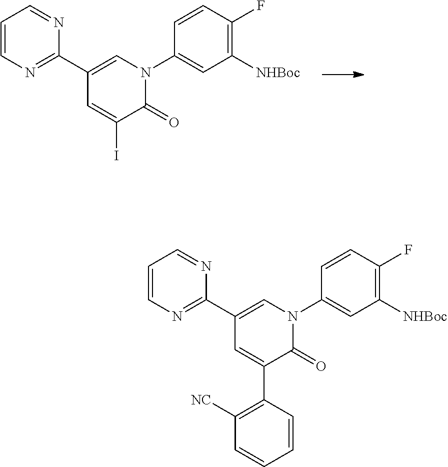 [SMILES: CC(C)(C)OC(=O)Nc1cc(-n2cc(-c3ncccn3)cc(I)c2=O)ccc1F ; CC(C)(C)OC(=O)Nc1cc(-n2cc(-c3ncccn3)cc(-c3ccccc3C#N)c2=O)ccc1F]

A mixture of the compound of Reference Example 16 (110 mg, 0.22 mmol), 2-(1,3,2-dioxaboran-2-yl)benzonitrile (81 mg, 0.43 mmol), cesium carbonate (212 mg, 0.65 mmol) and tetrakis(triphenylphosphine)palladium(0) (25 mg, 22 μmol) in DMF (10 mL) was stirred at 110° C. for 1 h under nitrogen atmosphere. The reaction mixture was diluted with water, was extracted with ethyl acetate and dried over anhydrous magnesium sulfate. The organic layer was evaporated in vacuo and the residue was purified by silica gel column chromatography using n-heptane/ethyl acetate (1/4, v/v) to give the title compound (71 mg, 68% yield).

¹H-NMR. (400 MHz, CDCl₃, δ): 1.52 (s, 9H), 6.83 (brs, 1H), 7.12-7.17 (m, 2H), 7.20 (dd, J=8.4, 10.4 Hz, 1H), 7.44 (t, J=7.6 Hz, 1H), 7.63 (t, J=7.6 Hz, 1H), 7.73 (d, J=7.6 Hz, 1H), 7.76 (d, J=7.6 Hz, 1H), 8.35 (brd, J=5.4 Hz, 1H), 8.68-8.73 (m, 2H), 8.70 (d, J=4.6 Hz, 2H).

ESI-MS: m/z 484 (M+H).

### Reference Example 19

### Synthesis of 2-(1-(3-amino-4-fluorophenyl)-2-oxo-5-(pyrimidin-2-yl)-1,2-dihydropyridin-3-yl)benzonitrile

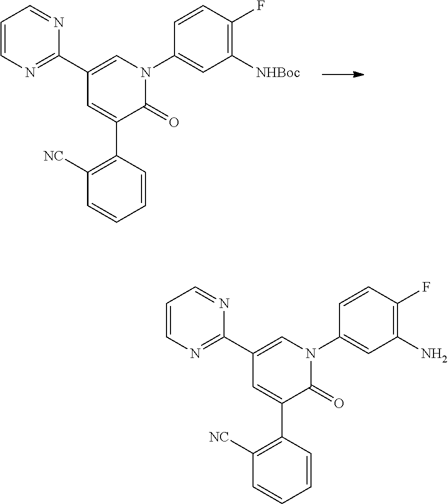 [SMILES: CC(C)(C)OC(=O)Nc1cc(-n2cc(-c3ncccn3)cc(-c3ccccc3C#N)c2=O)ccc1F ; N#Cc1ccccc1-c1cc(-c2ncccn2)cn(-c2ccc(F)c(N)c2)c1=O]

The compound of Reference Example 18 (70 mg, 0.14 mmol) was dissolved in trifluoroacetic acid (2 mL) at 0° C. and the mixture was stirred for 10 min. The excess amount of trifluoroacetic acid was removed in vacuo, the residue was neutralized with a saturated aqueous solution of sodium hydrogen carbonate, extracted with ethyl acetate and dried over anhydrous magnesium sulfate. The organic layer was evaporated in vacuo and the residue was purified by silica gel column chromatography using n-heptane/ethyl acetate (3/7, v/v) to give the title compound (43 mg, 77% yield).

¹H-NMR (400 MHz, CDCl₃, δ): 3.90 (brs, 2H), 6.80 (ddd, J=2.8, 4.0, 8.4 Hz, 1H), 6.95 (dd, J=2.4, 8.4 Hz, 1H), 7.11 (dd, J=8.4, 10.6 Hz, 1H), 7.15 (t, J=4.8 Hz, 1H), 7.45 (t, J=7.6 Hz, 1H), 7.63 (t, J=7.6 Hz, 1H), 7.70 (d, J=7.6 Hz, 1H), 7.76 (d, J=7.6 Hz, 1H), 8.69 (d, J=2.4 Hz, 1H), 8.70 (d, J=4.8 Hz, 2H), 8.71 (d, J=2.4 Hz, 1H).

ESI-MS: m/z 384 (M+H).

HRMS (FAB) calcd for C₂₂H₁₅FN₅O, 384.1261. found, 384.1292.

### Reference Example 20

### Synthesis of 3-bromo-1-(3-(hydroxymethyl)phenyl)-5-(pyrimidin-2-yl)pyridin-2(1H)-one

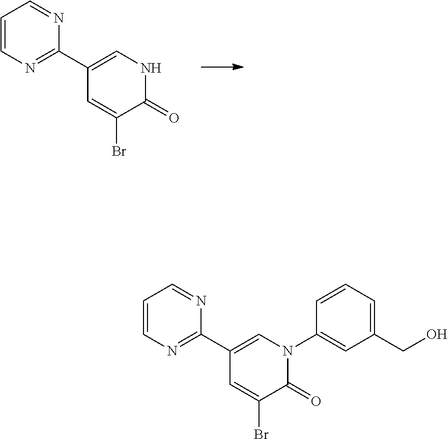 [SMILES: O=c1[nH]cc(-c2ncccn2)cc1Br ; O=c1c(Br)cc(-c2ncccn2)cn1-c1cccc(CO)c1]

A mixture of the compound of Reference Example 2 (300 mg, 1.2 mmol), 3-(hydroxymethyl)phenylboronic acid (543 mg, 3.6 mmol), copper(II) acetate (650 mg, 3.6 mmol) and pyridine (0.96 mL, 11.9 mmol) in DMF (5 mL) was stirred at 60° C. for 5 h. The reaction mixture was diluted with a 10% aqueous ammonia solution (50 mL) and stirred for 1.5 h. The resulting solid was filtered, rinsed with water, and collected. The solid was dried in vacuo to give the title compound (326 mg, 73% yield).

¹H-NMR (400 MHz, CDCl₃, δ): 1.97 (t, J=6.2 Hz, 1H), 4.78 (d, J=5.86 Hz, 2H), 7.16 (t, J=4.8 Hz, 1H), 7.37 (dt, J=7.6, 1.8 Hz, 1H), 7.45-7.54 (m, 3H), 8.63 (d, J=2.3 Hz, 1H), 8.70 (d, J=4.7 Hz, 2H), 8.91 (d, J=2.3 Hz, 1H).

ESI-MS: m/z 358 (M+H), 380 (M+Na).

### Reference Example 21

### Synthesis of 3-(2-fluorophenyl)-1-(3-(hydroxymethyl)phenyl)-5-(pyrimidin-2-yl)pyridin-2(1H)-one

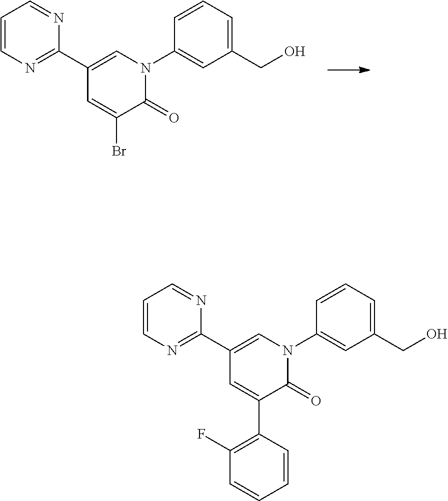 [SMILES: O=c1c(Br)cc(-c2ncccn2)cn1-c1cccc(CO)c1 ; O=c1c(-c2ccccc2F)cc(-c2ncccn2)cn1-c1cccc(CO)c1]

A mixture of the compound of Reference Example 20 (150 mg, 0.42 mmol), 2-fluorophenylboronic acid (117 mg, 0.83 mmol), cesium carbonate (409 g, 1.26 mmol) and tetrakis(triphenylphosphine)palladium(0) (24 mg, 21 μmol) in DMF (5 mL) was stirred at 120° C. for 2 h under nitrogen atmosphere. The reaction mixture was diluted with water, extracted with ethyl acetate and dried over anhydrous magnesium sulfate. The organic layer was evaporated in vacuo and the residue was purified by silica gel column chromatography using ethyl acetate to give the title compound (140 mg, 90% yield).

¹H-NMR (400 MHz, CDCl₃, δ): 4.78 (d, J=6.2 Hz, 2H), 7.13-7.16 (m, 2H), 7.16-7.22 (m, 1H), 7.30-7.38 (m, 1H), 7.42-7.48 (m, 2H), 7.51 (d, J=7.3 Hz, 1H), 7.57-7.55 (m, 1H), 7.62 (td, J=1.8, 7.2 Hz, 1H), 8.64 (dd, J=1.2, 2.6 Hz, 1H), 8.72-8.68 (m, 3H).

ESI-MS: m/z 374 (M+H).

HRMS (FAB) calcd for C₂₂FN₁₇FN₃O₂, 374.1305. found, 374.1290.

### Reference Example 22

### Synthesis of 3-(2-fluorophenyl)-1-(3-(fluoromethyl)phenyl)-5-(pyrimidin-2-yl)pyridin-2(1H)-one

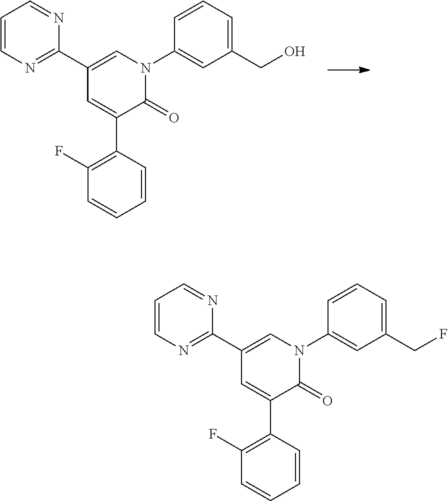 [SMILES: O=c1c(-c2ccccc2F)cc(-c2ncccn2)cn1-c1cccc(CO)c1 ; O=c1c(-c2ccccc2F)cc(-c2ncccn2)cn1-c1cccc(CF)c1]

Bis(2-methoxymethyl)aminosulfar trifluoride (22 μL, 0.12 mmol) was added to the solution of the compound of Reference Example 21 (15 mg, 0.04 mmol) in anhydrous dichloromethane (3 mL) at 0° C. under nitrogen atmosphere and stirred for 30 min. The reaction mixture was purified directly by silica gel column chromatography using ethyl acetate to give the title compound (12 mg, 82% yield).

¹H-NMR (400 MHz, CDCl₃, δ): 5.46 (d, J=47.6 Hz, 2H), 7.12-7.17 (m, 1H), 7.15 (t, J=4.8 Hz, 1H), 7.17-7.22 (m, 1H), 7.32-7.38 (m, 1H), 7.45-7.49 (m, 1H), 7.50-7.59 (m, 3H), 7.62 (td, J=1.8, 7.5 Hz, 1H), 8.64 (dd, J=1.2, 2.6 Hz, 1H), 8.69-8.72 (m, 3H).

ESI-MS: m/z 376 (M+H).

HRMS (FAB) calcd for C₂₂H₁₆F₂N₃O, 376.1261. found, 376.1281.

### Reference Example 23

### Synthesis of 3-(2-fluorophenyl)-1-(3-(p-toluenesulfonyloxymethyl)phenyl)-5-(pyrimidin-2-yl)pyridin-2(1H)-one

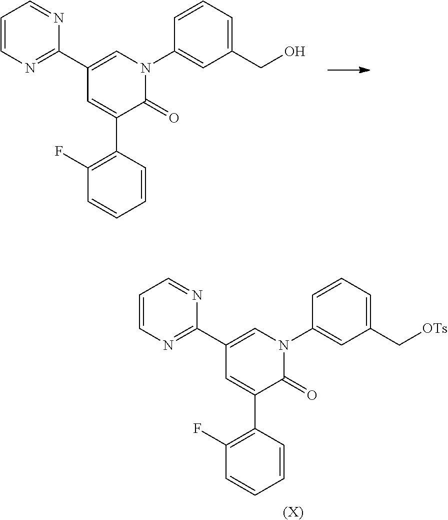

A mixture of the compound of Reference Example 21 (60 mg, 0.16 mmol), p-toluenesulfonyl chloride (80 mg, 0.42 mmol) in dichloromethane (3 mL) was added triethylamine (60 μL, 0.43 mmol)) and stirred at room temperature for 2 h under nitrogen atmosphere. The reaction mixture was diluted with water, was extracted with ethyl acetate and dried over anhydrous magnesium sulfate. The organic layer was evaporated in vacuo and the residue was purified by silica gel column chromatography using n-hexane/ethyl acetate (1/3, v/v) to give the title compound (25 mg, 29% yield). In addition, 35 mg of unreacted starting material was recovered.

¹H-NMR (300 MHz, CDCl₃, δ): 2.42 (s, 3H), 5.11 (s, 2H), 7.10-7.23 (m, 3H), 7.30-7.40 (m, 5H), 7.47-7.50 (m, 2H), 7.59 (td, J=1.8, 7.7 Hz, 1H), 7.81 (d, J=8.1 Hz, 2H), 8.60-8.59 (m, 2H), 8.71 (d, J=4.8 Hz, 2H).

HRMS (FAB) calcd for C₂₉H₂₃F₂N₃O₄S, 528.1393. found, 528.1360.

### Example 1

### Synthesis of 2-(1-(3-(methylamino)phenyl)-2-oxo-5-(pyrimidin-2-yl)-1,2-dihydropyridin-3-yl)benzo[¹¹C]nitile

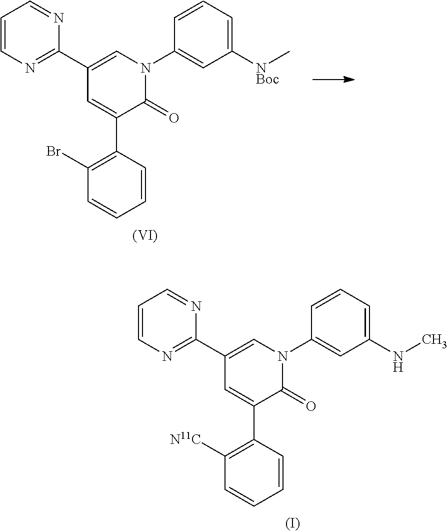

### [¹¹C]HCN Preparation;

Carbon-11 (¹¹C) was produced by the ¹⁴N(p,α)¹¹C nuclear reactions using CYPRIS HM-18 cyclotron (Sumitomo Heavy Industry, Tokyo). If not otherwise stated, radioactivity was measured with an IGC-3R Curiemeter (Aloka, Tokyo). In radio-HPLC purification and analysis, effluent radioactivity was monitored using a NaI (TI) scintillation detector system. [¹¹C]HCN was synthesized by a handmade device in a two-step sequence of reaction. After [¹¹C]CO₂ in nitrogen gas from the cyclotron was trapped at 196° C., it was heated to 50° C., moved under nitrogen stream (flow rate of 10 mL/min) and mixed with H₂ gas at a flow rate of 10 mL/min. The mixed gas was passed through a Ni wire tube at 400° C. in the methanizer to give a mixture of [¹¹C]CH₄ in the carrier gas. Then it was mixed with 5% NH₃ in nitrogen (v/v) gas stream at a flow rate of 400 mL/min and passed through Pt furnace at 950° C. to give a [¹¹C]HCN containing gas, which was absorbed to the reaction solution via a bubbling tube until the radioactivity of the vessel reached saturation (45 s). The average of the total radioactivity recovered in the reaction vessel was about 70% based on [¹¹C]CO₂ at end of synthesis (EOS). The synthesis of 2-(1-(3-(methylamino)phenyl)-2-oxo-5-(pyrimidin-2-yl)-1,2-dihydropyridin-3-yl)benzo[¹¹C]nitrile was successfully carried out by reaction with [¹¹C]CuCN, which was derived from [¹¹C]HCN shown in non-patent literatures 17-18.

A freshly prepared solution of sodium metabisulfite (150 μL, 48 mM; 7.2 μmol) was added to a solution of copper(II) sulfate (150 μL, 44 mM; 6.6 μmol) at room temperature under nitrogen stream 10 min prior to end of bombardment (EOB). [¹¹C]HCN gas was bubbled into the mixture at room temperature and a flow rate of 400 mL/min until the radioactivity reached saturation. The solution was then heated to 80° C. for 2 min. A solution of Reference Example 8 (3.3 mg, 7.4 μmol) in DMF (250 μL) was added to the reaction mixture at room temperature and heated to 165° C. for 3 min. Subsequently it was cooled to 80° C., Trifluoroacetic acid (500 μL) was added to the reaction mixture and heated at 80° C. for 3 min. The reaction mixture was allowed to cool to room temperature, then neutralized with a 5 M aqueous sodium acetate solution (1.25 mL) and purified by HPLC (Capcell Pack C18) using a mobile phase of acetonitile/water/triethylamine (5/5/0.01, v/v/v) at a flow rate of 5.0 mL/min to give 199.4 MBq of the title compound. The retention time (t_{R}) of the title compound was 11.0 min for purification and 5.8 min for analysis on HPLC. The synthesis time from EOB, 35.4 min; radiochemical yield (decay-corrected), 5.8% based on [¹¹C]CO₂; radiochemical purity, >99%; specific activity at EOS, 61 GBq/μmol. The analytical HPLC conditions are as follows: Column: Capcell Pack C₁₈, S-5 μm, 4.6 mm ID×250 mm; eluent: acetonitrile/water/triethylamine=5.5/4.5/0.01 (v/v/v); flow rate: 1.0 mL/min; Detector: UV-254 nm and RI.

### Example 2

### Synthesis of 2-(1-(3-amino-4-fluorophenyl)-2-oxo-5-(pyrimidin-2-yl)-1,2-dihydropyridin-3-yl)benzo[¹¹C]nitrile

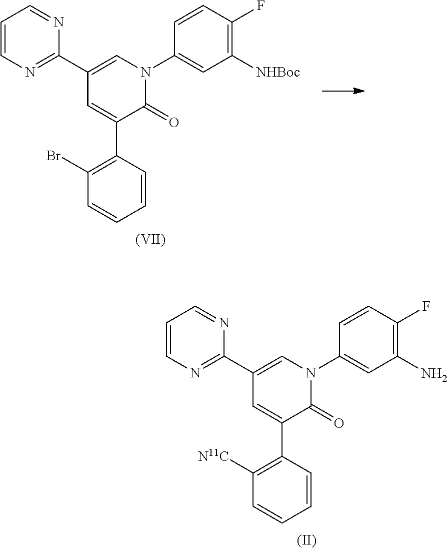

A freshly prepared solution of sodium metabisulfite (150 μL, 48 mM; 7.2 μmol) was added to a solution of copper(II) sulfate (150 μL, 44 mM; 6.6 μmol) at room temperature under nitrogen stream 10 min prior to EOB. [¹¹C]HCN gas was bubbled into the mixture at room temperature and a flow rate of 400 mL/min until the radioactivity reached saturation. The solution was then heated to 80° C. for 2 min. A solution of Reference Example 17 (3.54 mg, 6.8 μmol) in DMF (250 μL) was added to the reaction mixture at room temperature and heated to 165° C. for 3 min. The reaction mixture was cooled to 80° C., trifluoroacetic acid (0.5 mL) was added to the reaction mixture and heated at 80° C. for 3 min. The reaction mixture was neutralized with a 5 M aqueous sodium acetate solution (1.25 mL), then was purified by HPLC (Capcell Pack C₁₈) using a mobile phase of acetonitrile/water/triethylamine (4.5/5.5/0.01, v/v/v) at a flow rate of 5.0 mL/min to give the title compound (1.52 GBq yield at EOS from 37.4 GBq of bombardment at EOB). The t_{R} of the title compound was 11.0 min for purification and 9.2 min for analysis on HPLC. The synthesis time from EOB, 37.0 min; radiochemical yield (decay-corrected), 14.3% based on [¹¹C]CO₂; radiochemical purity, >99%; specific activity at EOS, 194 GBq/μmol. The analytical HPLC conditions are as follows: Column: Capcell Pack C₁₈, S-5 μm, 4.6 mm ID×250 mm; eluent: acetonitrile/water/triethylamine=4.5/5.5/0.01 (v/v/v); flow rate: 1.0 mL/min; Detector: UV-254 nm and RI.

### Example 3

### Synthesis of 2-(1-(3-fluorophenyl)-2-oxo-5-(pyrimidin-2-yl)-1,2-dihydropyridin-3-yl)benzo[¹¹C]nitrile

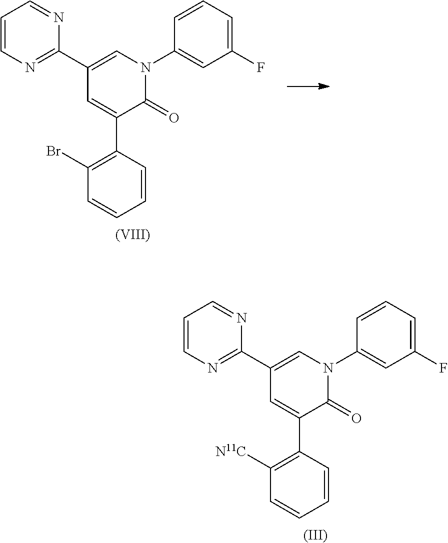

A freshly prepared solution of sodium metabisulfite (150 μL, 48 mM; 7.2 μmol) was added to a solution of copper(II) sulfate (150 μL, 44 mM; 6.6 μmol) at room temperature under nitrogen stream 10 min prior to EOB. [¹¹C]HCN gas was bubbled into the mixture at room temperature and a flow rate of 400 mL/min until the radioactivity reached saturation. The solution was then heated to 80° C. for 2 min. A solution of Reference Example 14 (3.42 mg, 8.1 μmol) in DMF (250 μL) was added to the reaction mixture and heated to 165° C. for 5 min. The reaction mixture was then purified by HPLC (Capcell Pack C₁₈) using a mobile phase of acetonitrile/water/triethylamine (5.0/5.0/0.01, v/v/v) at a flow rate of 5.0 mL/min to give the title compound (2.54 GBq yield at EOS from 35.5 GBq of bombardment at EOB). The t_{R} of the title compound was 12.1 min for purification and 6.1 min for analysis on HPLC. The synthesis time from EOB, 36.0 min; radiochemical yield (decay-corrected), 24.6% based on [¹¹C]CO₂; radiochemical purity, >99%; specific activity at EOS, 91 GBq/μmol. The analytical HPLC conditions are as follows: Column: Capcell Pack C₁₈, S-5 μm, 4.6 mm ID×250 mm; eluent: acetonitrile/water/triethylamine=7.0/3.0/0.01 (v/v/v); flow rate: 1.0 ml/min; Detector. UV-254 nm and RI.

### Example 4

### Synthesis of 2-(1-(3-([¹¹C]methylamino)phenyl)-2-oxo-5-(pyrimidin-2-yl)-1,2-dihydropyridin-3-yl)benzonitrile

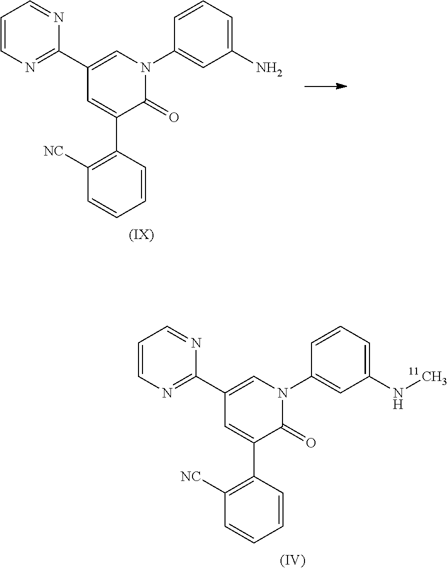

[¹¹C]Methyl Trifluoromethanesulfonate (MeOTf) Preparation;

[¹¹C]MeOTf was synthesized as a procedure shown in non-patent literatures 19-22. [¹¹C]MeOTf was generated by a reaction of the produced [¹¹C]CH₃I with 150-200 mg of silver triflate (fixed on Graphpac GC; quartz glass column; I.D.: 3.9 mm; O.D.: 6 mm; length: 200 mm) in an online flowthrough process at 180° C. using a nitrogen gas flow of 50 mL/min.

[¹¹C]MeOTf gas was introduced to Reference Example 6 (0.50 mg, 1.3 μmol) in dry acetone (250 μL) through bubbling tube at room temperature until the radioactivity reached saturation and subsequently the reaction mixture was dried over at 80° C. under nitrogen stream. The residue was dissolved with HPLC eluent and was purified by HPLC (Capcell Pack C₁₈) using a mobile phase of acetonitrile/water/triethylamine (4.5/5.5/0.01, v/v/v) at a flow rate of 5.0 mL/min to give the title compound (1.69 GBq yield at EOS from 21.4 GBq of bombardment at EOB). The synthesis time from EOB, 32.4 min; radiochemical yield (decay-corrected), 23.7% based on [¹¹C]CO₂; radiochemical purity, >99%; specific activity at EOS, 183 GBq/μmol. The analytical HPLC conditions are as follows: Column: Capcell Pack C₁₈, S-5 μm, 4.6 mm ID×250 mm; eluent: acetonitrile/water/triethylamine=5.5/4.5/0.01 (v/v/v); flow rate: 1.0 mL/min; Detector: UV-254 nm and RI. Additionally, very small amount of ¹¹C-labeled Reference Example 11 was detectable on HPLC analysis.

### Example 5

### Synthesis of 3-(2-fluorophenyl)-1-(3-([¹⁸F]fluoromethyl)phenyl)-5-(pyrimidin-2-yl)pyridin-2(1H)-one

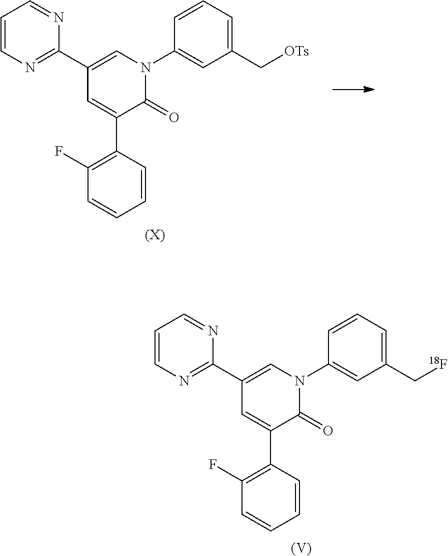

[¹⁸F]F⁻ preparation; [¹⁸F]F⁻ was produced by the ¹⁸O (p,n)¹⁸F reaction on 95 atom % H₂¹⁸O using 18 MeV protons (14.2 MeV on target) from the cyclotron and separated from [¹⁸O]H₂O using Dowex 1-X8 anion exchange resin. The [¹⁸F]F⁻ was eluted from the resin with aqueous solution of potassium carbonate (10 mM, 500 μL) into a vial containing a solution of 4,7,13,16,21,24-hexaoxa-1,10-diazabicyclo[8,8,8]hexacosane (Kryptofix 222, 25 mg) in acetonitrile (1.5 mL) and transferred into another reaction vessel in the hot cell. The [¹⁸F]F⁻ solution was dried to remove water and acetonitrile at 120° C. for 15 min.

A solution of Reference Example 23 (1.5 mg, 2.8 μmol) in anhydrous acetonitrile (300 μL) was added to the reaction vessel containing [¹⁸F]F⁻ and heated at 85° C. for 10 min. The reaction mixture was purified by HPLC (YMC, J'Sphere C₁₈, S-5 μm, 10 mm ID×250 mm) using a mobile phase of acetonitrile/water/triethylamine (5.0/5.0/0.01, v/v/v) at a flow rate of 5.0 mL/min to give the title compound (1.24 GBq yield at EOS from 5.00 GBq of bombardment at EOB). The t_{R} of the title compound was 17.3 min for purification and 6.5 min for analysis on HPLC. The synthesis time from EOB, 70.5 min; radiochemical yield (decay-corrected), 33.8% based on [¹⁸F]F⁻; radiochemical purity, >99%; specific activity at EOS, 297 GBq/μmol. The analytical HPLC conditions are as follows: Column: Capcell Pack C₁₈, S-5 μm, 4.6 mm ID×250 mm; eluent: acetonitrile/water/triethylamine=6.5/3.5/0.01 (v/v/v); flow rate: 1.0 mL/min; Detector: UV-254 nm and RI.

### Test Example 1

### Purity Measurement of the Compounds of Examples 1-5

The identities of the compound of Examples 1-5 were confirmed by co-injection with the corresponding unlabeled compounds on reverse phased-analytical HPLC. Specifically, the compound of Reference Example 9 corresponds to the compounds of Examples 1 and 4, the compound of Reference Example 19 corresponds to the compound of Example 2, the compound of Reference Example 15 corresponds to the compound of Example 3, and the compound of Reference Example 22 corresponds to the compound of Example 5. In the final product solutions, their radiochemical purities were higher than 99%. Additionally, specific activity of each product was calculated from the UV absorption area at 254 nm based on standard curves from known-concentrations of unlabeled compounds in common ratio. The amount of carrier in the final product solution was measured by the same analytical HPLC. Moreover, these radioligands, the compounds of Examples 1-5, did not show radiolysis at room temperature for 90 min after formulation, indicating radiochemical stability over the period of at least one PET scan.

All labeled compounds were formulated with sterile saline (3 mL) containing Tween (trademark) 80 (100 μL) and ascorbic acid (25 mg) after HPLC purification.

### Test Example 2

### Kᵢ Value Measurement. (In Vitro Receptor Binding Assay)

### Preparation of Receptor Solution,

Homogenate of forebrains of rats (Sprague-Dawley) was prepared in ice-cold solution containing 0.32 M sucrose and 0.1 mM EGTA (pH 7.4). Homogenate was centrifuged in 1000 g for 10 min and supernatant was collected. This supernatant was centrifuged in 30000 g for 20 min. Precipitate was suspended in 1 mM EGTA/Tris buffer (pH 8.0) by sonication, subjected osmotic lysis on ice for 10 min and centrifuged in 30000 g for 20 min. This procedure was conducted twice. Precipitate was suspended in 50 mM Tris HCl buffer (pH 7.4) by sonication and centrifuged in 30000 g for 20 min. This procedure was conducted three times. Precipitate was suspended in 50 mM Tris HCl buffer (pH 7.4) by sonication and stocked at −80° C. On the day of binding assay, stocked solution was suspended in 50 mM Tris HCl buffer (pH 7.4) by sonication and centrifuged in 30000 g for 20 min. This procedure was conducted three times. Precipitate was suspended in 50 mM Tris-HCl buffer (pH 7.4) by sonication and used for binding assay.

### Binding Assay;

Receptor solution was re-suspended in binding buffer (50 mM Tris-HCl, pH 7.4) to a final concentration of 0.24 mg tissue eq./assay. The incubation time for [³H]perampanel on AMPA receptor was 90 min at 4° C. After incubation, membranes were filtered onto GF/B filter presoaked with 0.3% PEI and washed three times with ice-cold wash buffer (same as binding buffer). Each filter was placed in a vial and 6 mL of liquid scintillator reagent (Hionic-Fluor; PerkinElmer Life & Analytical Sciences) were added. Radioactivity was counted (1 min) in a liquid scintillation counter (LSC-6100, Hitachi Aloka Medical, Ltd.).

Saturation isotherms were determined by addition of various concentrations of [³H]perampanel (1-2000 nM). Nonspecific bindings for [³H]perampanel was measured in the presence of 15 μM unlabeled compounds. Kᵢ value was calculated by Scatchard analysis of saturation isotherm experiment. The results are shown in the following table.

| | Kᵢ value (nM) |
|---|---|
| Reference Example 9 which | 10 |
| corresponds to Examples 1 and 4 | |
| Reference Example 19 which | 5 |
| corresponds to Example 2 | |
| Reference Example 15 which | 20 |
| corresponds to Example 3 | |
| Reference Example 22 which | 22 |
| corresponds to Example 5 | |

### Test Example 3

### In Vitro Autoradiography Using Rat Brain Sections

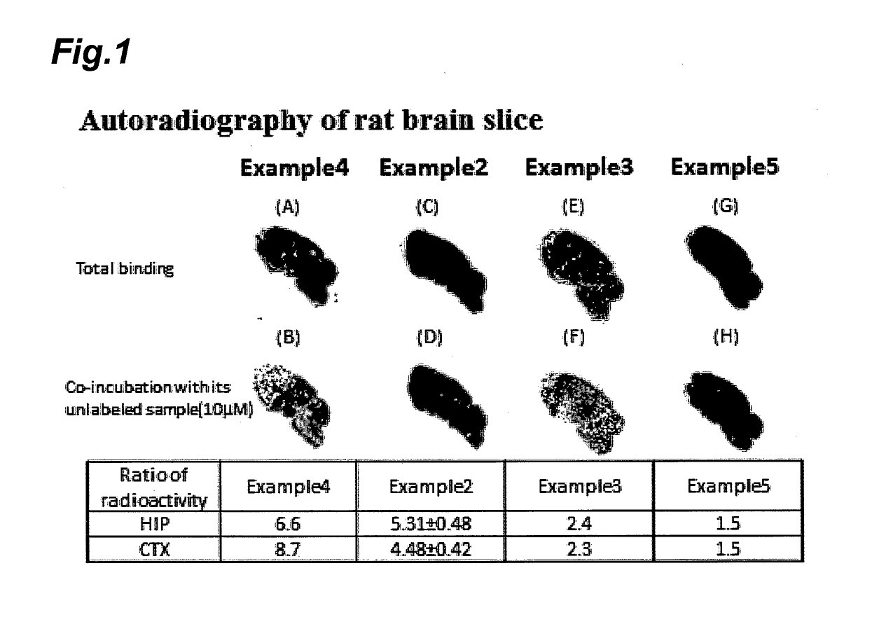

Rat brain sections (20 μm-thick) were dried up at room temperature and pre-incubated for 20 min in 50 mM Tris-HCl buffer (pH 7.4) containing 2.5 mM calcium chloride at 4° C. After pre-incubation, these sections were incubated for 60 min at 4° C. in fresh buffer with appropriate concentration of the compounds of Examples 1-5 (1-10 nM), respectively. Unlabeled compounds (10 μM) were used to assess the nonspecific binding of these radioligands in the brain. After incubation, brain sections were rapidly washed twice with assay buffer which is same as used in incubation, and dried up at room temperature. An imaging plate (BAS-IP MS2025, Fujifilm, Tokyo, Japan) was exposed to the dried sections for 1 h. Radioactive standards calibrated with known amounts of the labeled compounds were induced in the exposure process. Quantitative autoradiogram analysis was performed using a computer-assisted image analyzer (Multi Gauge; Fujifilm). Optical density values were converted to fmol/mg protein using a computer generated regression analysis which compared film densities produced by tissue sections and radioactive standards. The results are shown in FIG. 1. Ratios of radioactivities between slices untreated and treated with unlabeled compound in neocortex (CTX) and hippocampus (HIP) were calculated. All tested radioligands exhibited detectable specific binding to AMPA receptor especially in CTX, HLP, and striatum (STR), in line with known distribution of AMPA receptor.

### Test Example 4

### In Vitro Autoradiography Using Monkey Brain Sections

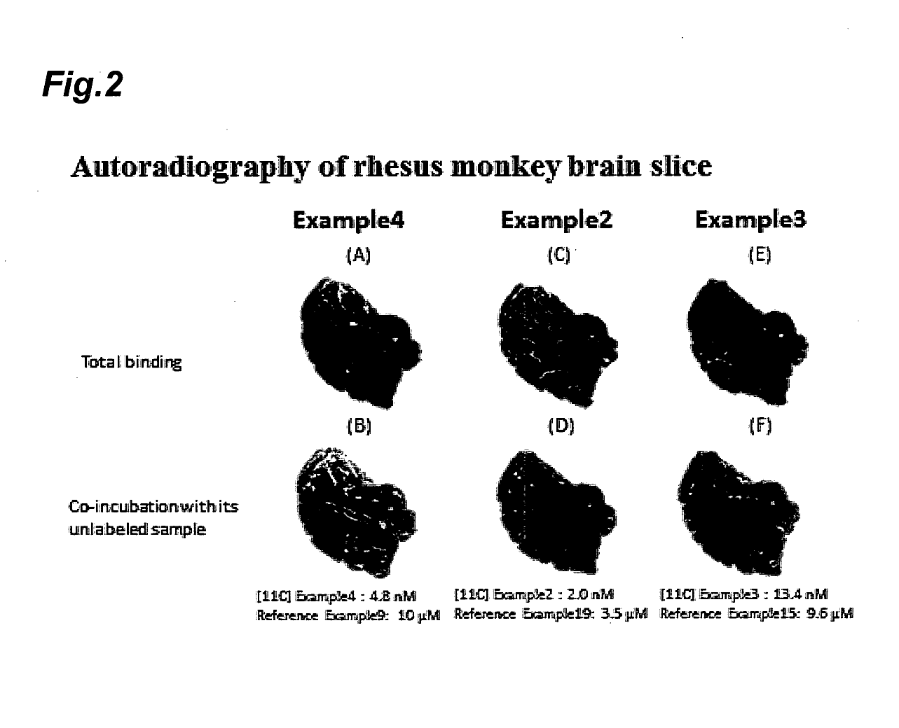

Similarly to Test Example 3, autoradiography was performed using monkey brain sections (20 μm-thick) instead of rat brain sections. The results are shown in FIG. 2.

All tested radioligands exhibited detectable specific binding to AMPA receptor especially in CTX, HIP, and STR, these signals were very similar to those of rat brain slice.

### Test Example 5

### In Vivo PET Study in Monkey

### Animals;

Rhesus monkeys at 3 years and 8 months of age (male, 4.25 kg, 4.50 kg weight, respectively) were purchased from Japan SLC (Shizuoka, Japan). The monkeys were housed in an individual cage and were supplied with a balanced diet and ad libitum tap water from a feeding valve. The room was illuminated from 7 a.m. to 9 p.m. Age and body weight of the monkey at that time of PET scan was 3 years and 11 months and approximately 5 to 6 kg, respectively. The animal experiments were approved by the Animal Ethics Committee of National Institute of Radiological Sciences (NIRS).

### MRI for Monkey;

Prior to PET scans, anatomical template images of the monkey brain were generated by a high-resolution MRI system. Briefly, a monkey was anesthetized with sodium pentobarbital (50 mg/kg, i.p.), and scanned with a 400 mm bore, 7 Tesla horizontal magnet (NIRS/KOBELCO, Kobe, Japan/Bruker BioSpin) equipped with 120 mm diameter gradients (Bruker BioSpin). A 72 mm diameter coil was used for radiofrequency transmission, and signals were received by a 4-channel surface coil. Coronal T2-weighted MRI images were obtained by a fast spin-echo sequence termed FLASH with the following imaging parameters: repetition time=481 msec, effective echo time=7.6 msec, FOV=110 mm×110 mm, slice thickness=1.5 mm.

### PET Scans for Monkey;

PET scans for a monkey was performed using a high-resolution SHR-7700 PET camera (Hamamatsu Photonics, Shizuoka, Japan) designed for laboratory animals, which provides 31 transaxial slices 3.6 mm (center-to-center) apart and a 33.1 cm (transaxial)×11.16 cm (axial) FOV. The spatial resolution for the reconstructed images was 2.6 mm FWHM at the center of FOV. Prior to PET scans, the monkey was initially anesthetized with thiamylal and anesthesia was maintained using 1.5% (v/v) isoflurane. Following a transmission scan for attenuation correction using a ⁶⁸Ge-⁶⁸Ga source, dynamic emission scans were conducted in a 3-dimensional acquisition mode for 90 min (frames, 1 min×4 scans, 2 min×8 scans, 5 min×14 scans). Emission scan images were reconstructed with a 4 mm Colsher filter. A radioligand was injected via the crural vein as a single bolus at the start of emission scanning. Injected dose of the radioligand was approximate 110 MBq/head.

### Image Acquisition and Data Analysis.

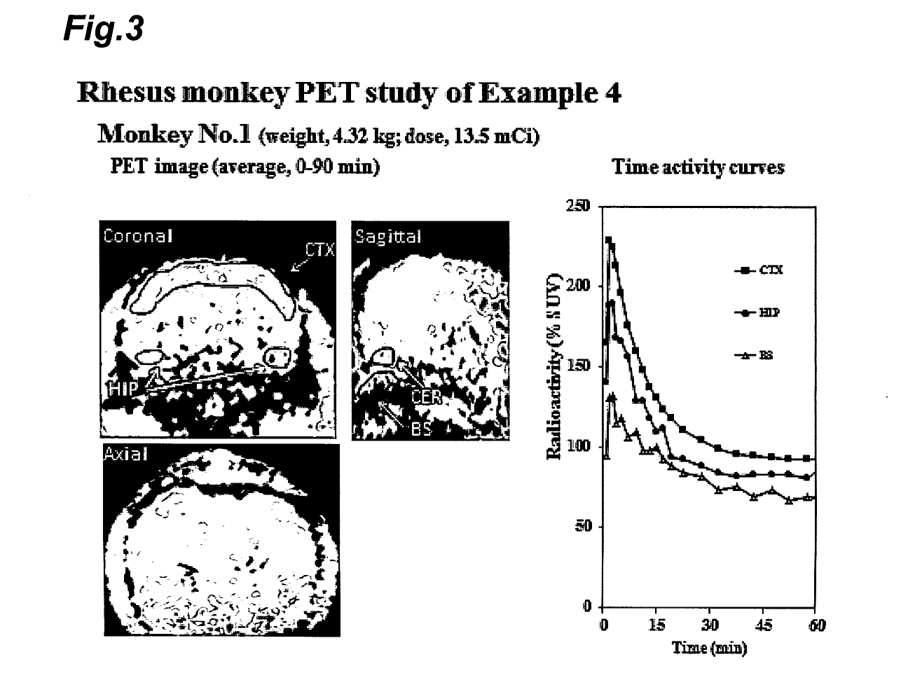

Anatomical regions of interest (ROIs) were manually defined on the striatum, thalamus, pons, and cerebellum in the PET images coregistered with MRI images using PMOD (trademark) software (PMOD Technologies Ltd., Zurich, Switzerland). Regional radioactivity in the brain was decay-corrected to the injection time and was expressed as the percent of injected dose (% ID/mL=% injected dose/cm³ brain) in rat experiments, and as the percentage standardized uptake value [% SUV=% ID/mL×body weight (g)] in monkey experiments. The results are shown in FIG. 3.

The uptake of radioactivity in all brain regions peaked at 30 s after intravenous radioligand injection, followed by a rapid decline of radioactivity and moderate level retention in the brain. Notable retention of radioactivity in AMPA receptor-rich brain regions in HIP and CTX, in contrast with low uptake in the brain stem (BS) was observed. These data are consistent with in vitro autoradiographic image shown in Test Examples 3 and 4.

### Test Example 6

### In Vivo PET Blocking Study in Monkey

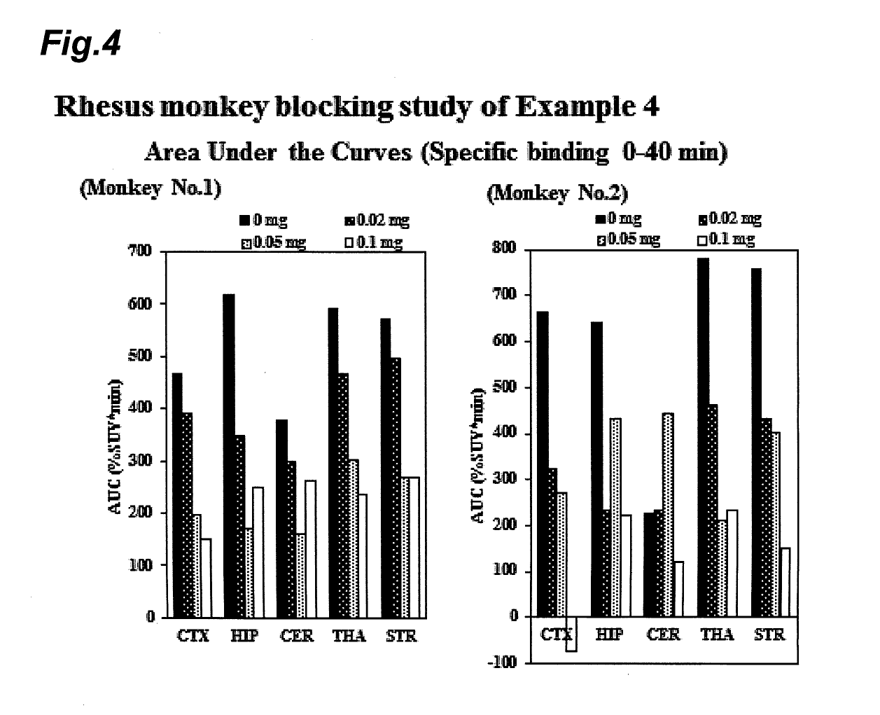

For the PET blocking study, unlabeled compound (Reference Example 9, 0.1 mg/kg/5 mL to 0.02 mg/kg/5 mL of saline with 10% DMSO, 3 doses) was intravenously administrated via the crural vein as a single slow bolus over 10 min, 15 min prior to radioligand administration. All PET scans were acquired from the same two monkeys (n=2). The results are shown in FIG. 4.

Area under time-radioactivity curves of to the region of interests (CTX, HIP, cerebellum (CER), thalamus (THA), STR) in the absence (control) and blockade by pretreatment with Reference Example 9. Radioactivity is expressed as percentage of standardized uptake value (% SUV) and integrated radioactivity from 0 to 40 min during the PET scan. Pretreatment with Reference Example 9 markedly reduced the radioactivity in dose dependent manner compared to control. Radioligand retention was significantly inhibited in all brain regions, and the distribution of radioactivity fairly uniform throughout the brain.

1. A compound selected from the group consisting of
2-(1-(3-methylaminophenyl)-2-oxo-5-(pyrimidin-2-yl)-1,2-dihydropyridin-3-yl)benzo[¹¹C]nitrile (I),
2-(1-(3-amino-4-fluorophenyl)-2-oxo-5-(pyrimidin-2-yl)-1,2-dihydropyridin-3-yl)benzo[¹¹C]nitrile (II),
2-(1-(3-fluorophenyl)-2-oxo-5-(pyrimidin-2-yl)-1,2-dihydropyridin-3-yl)benzo[¹¹C]nitrile (III),
2-(1-(3-[¹¹C]methylaminophenyl)-2-oxo-5-(pyrimidin-2-yl)-1,2-dihydropyridin-3-yl)benzonitrile (IV), and
3-(2 fluorophenyl)-1-(3-([¹⁸F]fluoromethyl)phenyl)-5-(pyrimidin-2-yl)pyridin-2(1H)-one (V)
or a pharmaceutically acceptable salt thereof.

2. The compound or pharmaceutically acceptable salt of claim 1 which is 2-(1-(3-methylaminophenyl)-2-oxo-5-(pyrimidin-2-yl)-1,2-dihydropyridin-3-yl)benzo[¹¹C]nitrile (I).

3. The compound or pharmaceutically acceptable salt of claim 1 which is 2-(1-(3-amino-4-fluorophenyl)-2-oxo-5-(pyrimidin-2-yl)-1,2-dihydropyridin-3-yl)benzo[¹¹C]nitrile (II).

4. The compound or pharmaceutically acceptable salt of claim 1 which is 2-(1-(3-fluorophenyl)-2-oxo-5-(pyrimidin-2-yl)-1,2-dihydropyridin-3-yl)benzo[¹¹C]nitrile (III).

5. The compound or pharmaceutically acceptable salt of claim 1 which is 2-(1-(3-[¹¹C]methylaminophenyl)-2-oxo-5-(pyrimidin-2-yl)-1,2-dihydropyridin-3-yl)benzonitrile (IV).

6. The compound or pharmaceutically acceptable salt of claim 1 which is 3-(2-fluorophenyl)-1-(3-([¹⁸F]fluoromethyl)phenyl)-5-(pyrimidin-2-yl)pyridin-2(1H)-one (V).

7. A composition comprising the compound or pharmaceutically acceptable salt of claim 1 as an active ingredient and a vehicle.

8. A PET probe comprising the compound or pharmaceutically acceptable salt of claim 1 and a vehicle.

9. The PET probe of claim 8 for imaging AMPA receptor.

10. A method of imaging comprising administering the compound or pharmaceutically acceptable salt of claim 1 to a subject, and visualizing the compound or salt in the subject by PET.

11. The method of claim 10, for imaging AMPA receptor.

12.-15. (canceled)
